(19) United States
(12) Patent Application Publication
(10) Pub. No.: US 2026/0345694 A1
(43) Pub. Date: Nov. 5, 2026

(54) COMPOSITIONS AND METHODS TO IMPROVE INTESTINAL INTEGRITY, BODY WEIGHT GAIN, AND/OR MILK PRODUCTION IN LIVESTOCK AND POULTRY

(71) Applicant: NutriQuest, LLC, Mason City, IA (US)

(72) Inventors: Sarah Elizabeth Schuling, Mason City, IA (US); Daniel Edward Schimek, Mason City, IA (US)

(21) Appl. No.: 19/378,097

(22) Filed: Nov. 3, 2025

Related U.S. Application Data
(60) Provisional application No. 63/799,187, filed on May 2, 2025.

(51) Int. Cl. A61K 35/742; A23K 10/18; A23K 20/111; A23K 50/10; A23K 50/30; A23K 50/60; A61K 31/216; A61K 36/74; A61P 1/00; A61P 15/14; A61P 29/00

(52) U.S. Cl. CPC A61K 35/742; A23K 10/18; A23K 20/111; A23K 50/10; A23K 50/30; A23K 50/60; A61K 31/216; A61K 36/742; A61P 1/00; A61P 15/14; A61P 29/00

(57) ABSTRACT

A Bacillus and chlorogenic acid composition and its method of use are provided. The composition and method of use function to improve intestinal integrity, body weight gain, and milk production in livestock and poultry.

### FIELD OF THE INVENTION

The present disclosure generally relates to a livestock feed additive composition. More particularly, the present disclosure is concerned with a feed additive composition comprising at least one strain of bacteria from the Bacillus genus and chlorogenic acid. The present disclosure also relates to the use of such compositions to improve intestinal integrity, body weight gain, and milk production in livestock and poultry.

### BACKGROUND OF THE INVENTION

Intestinal integrity refers to the ability of the intestine to maintain its structural and functional soundness. A healthy and unimpaired intestine is critical for the efficient operation of the gastrointestinal tract (GIT). A well-functioning GIT ensures effective digestion and absorption of nutrients, reduced nutrient wastage, resistance against entero-pathogens, lower morbidity and mortality rates, and optimized feed conversion ratio.

The intestine employs a physical barrier to prevent the entry of foreign materials and pathogens into the bloodstream or access to other internal organs. The gut epithelial barrier is comprised of epithelial cells, mucus, and tight junction proteins (TJ proteins). The TJ proteins form a barrier to seal the gaps between the epithelial cells to prevent the leakage of larger unwanted molecules into the bloodstream. When TJ proteins are compromised and no longer intact, molecules such as lipopolysaccharide (LPS) can enter the bloodstream and cause a physiological response from the animal. LPS is an important outer membrane component of gram-negative bacteria. Once absorbed into the animal, an immune response occurs to clear LPS from the body.

Compromised intestinal integrity (commonly known as leaky gut) can occur in livestock and poultry species due to a number of different factors, including: degradation of or damage to the physical barriers as a result of pathogens such as viruses, bacteria, fungi, parasites, and toxins; environmental stressors such as handling, transportation, overcrowding, and abrupt environmental changes; feed toxins; dietary factors such as nutritional imbalances, microbial contamination of feed, and the presence of non-starch polysaccharides (NSP) in diets; diseases, infections, and injuries; and gut microflora.

To prevent leaky gut in animal production systems, ideally stressors are to be avoided to the extent possible. However, in modern production systems, stressors such as heat or extreme cold, off-feed events for example during transportation, and social stress such as overcrowding cannot be avoided. Maintaining intestinal integrity during unavoidable periods of stress can be beneficial to overall animal well-being and productivity.

In addition, improving milk production and lactation of cows and other non-human animals are also areas of interest in the livestock industry. Often during early lactation an animal experiences various negative health events including metabolic disorders such as hypocalcemia, ketosis, retained placenta. These disorders or other stressors can impact milk yield, frequency of milking, milk component concentration, and other aspects of the milking process. A product that helps to prevent and/or alleviate the impacts of these conditions would be beneficial to the entire milk production industry as well as have other positive downstream effects on the livestock industry in general.

The use of Bacillus genus as a probiotic has strong antimicrobial, antidiarrheal, and immunostimulatory effects in both humans and animals. Bacillus strains have also been reported to have anti-inflammatory properties without directly impacting expression of tight junction proteins in the intestine. Chlorogenic acid (CGA) is one of the major components of Green Coffee Bean (GCB) extract. Green coffee bean contains on average 26.8% CGA. It is well established that pure CGA improves intestinal barrier function in poultry and mammalian species. However, a highly purified source of CGA is not available. Improvement of intestinal barrier function with supplementation of GCB in animals is known to a lesser extent. One trial did report on improvement in intestinal barrier integrity with GCB supplementation in rats.

Independently, certain Bacillus strains and CGA (a component of GCB) have been reported to separately bring about positive effects on intestinal integrity and milk production across species. However, the impact and synergistic effect stemming from the combination of Bacillus strains with GCB extract on intestinal integrity, biomarkers of inflammation, animal performance, and lactation has not been previously reported.

Accordingly, there is an unmet need in the market for compositions and methods for improving intestinal integrity, lactation and milk production in livestock and other non-human animals. This disclosure aims to provide a novel composition and method to improve intestinal integrity, lactation, and milk production through supplementation with an effective combination of a strain of Bacillus and chlorogenic acid.

The present disclosure provides a composition used to improve intestinal integrity, body weight gain, milk production, and other health improvements in non-human animals. The feed or feed additive can be used for its beneficial effects in healthy non-human animals, or in response to a stressor such as off-feed events, heat, social interactions and many others. The composition disclosed is a therapeutically effective combination of a strain of Bacillus and a source of chlorogenic acid to be used as a feed supplement or as feed.

The composition is used in healthy and stressed animals to improve intestinal integrity, increase weight gain, decrease inflammation, increase milk yield, increase milk component yield, reduce negative health events in lactating animals, among other health benefits. The composition uses a variety of strains of Bacillus and the chlorogenic acid can come from a multitude of sources. However, green coffee beans and extracts thereof are the most common source of the chlorogenic acid in the composition.

### BRIEF DESCRIPTION OF THE FIGURES

The following drawings form part of the present specification and are included to further demonstrate certain aspects of the present disclosure. Certain aspects can be better understood by reference to one or more of these drawings in combination with the detailed description of the specific aspects presented herein.

FIG. 1 is a graph displaying the total fecal myeloperoxidase concentrations from Example 1 showing a 24-hour feed restriction event impacts tight junction proteins and physiological markers of inflammation. A combination of 2 Bacillus amyloliquefaciens strains fed at 500,000 CFU/g partially alleviated the negative impact of the feed restriction stress event.

FIG. 2 is a graph displaying the body weight by week of experiment from Example 1 showing a 24-hour feed restriction event impacts tight junction proteins and physiological markers of inflammation. A combination of 2 Bacillus amyloliquefaciens strains fed at 500,000 CFU/g partially alleviated the negative impact of the feed restriction stress event.

FIG. 3 is a graph displaying the change in lipopolysaccharide binding protein from Example 1 showing a 24-hour feed restriction event impacts tight junction proteins and physiological markers of inflammation. A combination of 2 Bacillus amyloliquefaciens strains fed at 500,000 CFU/g partially alleviated the negative impact of the feed restriction stress event.

FIG. 4 is a graph showing the relationship between plasma lipopolysaccharide binding protein concentrations and ileal expression of occludin from Example 1 showing a 24-hour feed restriction event impacts tight junction proteins and physiological markers of inflammation. A combination of 2 Bacillus amyloliquefaciens strains fed at 500,000 CFU/g partially alleviated the negative impact of the feed restriction stress event.

FIG. 5 is a graph displaying immunohistochemistry staining intensity for occludin from Example 1 showing a 24-hour feed restriction event impacts tight junction proteins and physiological markers of inflammation. A combination of 2 Bacillus amyloliquefaciens strains fed at 500,000 CFU/g partially alleviated the negative impact of the feed restriction stress event.

FIG. 6 is a graph showing the body weight by day of experiment from Example 2 showing that feeding green coffee bean extract in combination with Bacillus amyloliquefaciens species fed at 500,000 CFU/g reduced the negative impact of 24-hour feed restriction on ileal expression of tight junction proteins and markers of inflammation.

FIG. 7 depicts a graph showing the level of LPS binding protein 24 hours after feed restriction from Example 2 showing that feeding green coffee bean extract in combination with Bacillus amyloliquefaciens species fed at 500,000 CFU/g reduced the negative impact of 24-hour feed restriction on ileal expression of tight junction proteins and markers of inflammation.

FIG. 8 depicts a graph showing the transepithelial electrical resistance in IEC-6 rate intestinal cells at 6 hours from Example 3 showing that green coffee bean extract in combination with Bacillus amyloliquefaciens species increases transepithelial electrical resistance in IEC-6 rat intestinal cells.

FIG. 9 depicts a graph showing the average daily gain 4 days following a feed restriction event in Example 4 showing that green coffee bean extract in combination with Bacillus velezensis species increases average daily gain and alters markers of inflammation following a feed restriction stress event.

FIG. 10 depicts a graph showing the change in lipopolysaccharide binding protein following a 24-hour feed restriction event in Example 4 showing that green coffee bean extract in combination with Bacillus velezensis species increases average daily gain and alters markers of inflammation following a feed restriction stress event.

### DETAILED DESCRIPTION

The present disclosure is based on the discovery that administering a feed composition comprising a combination of at least one Bacillus strain and a form of chlorogenic acid to a non-human animal improves intestinal integrity, milk production, and lactation.

Additional aspects and features are set forth in part in the description that follows, and in part will become apparent to those skilled in the art upon examination of the specification or may be learned by the practice of the aspects discussed herein. A further understanding of the nature and advantages of certain aspects may be realized by reference to the remaining portions of the specification the drawings, the chemical structures, and descriptions, which forms a part of this disclosure. Any description of any R-group or chemical substituent, alone or in any combination, may be used in any chemical Formula described herein, and Formulae include all conformational and stereoisomers, including diastereomers, epimers, and enantiomers. Moreover, any feature of a composition disclosed herein may be used in combination with any other feature of a composition disclosed herein.

In some aspects, the composition may include the Bacillus amyloliquefaciens strains BA22 and BA1013 and chlorogenic acid in the form of green coffee bean extract. The dose range for the Bacillus 50:50 combination is 1×10⁴-3×10¹⁰ CFU/head/day and the dose range for the green coffee bean extract is 0.005-20 g/head/day. The preferred range for Bacillus is 5.0×10⁵-2×10⁹ CFU/head/day and for green coffee bean is 6-400 mg/head/day.

In certain aspects, the Bacillus strain is selected from a group of species consisting of Bacillus subtilis, Bacillus velezensis, Bacillus amyloliquefaciens, or a combination thereof.

In other aspects, the Bacillus species is selected from any of the species that fall under the genus of Bacillus, or a combination thereof.

In some aspects, chlorogenic acid may be in the form of ingredients that contain detectable levels of chlorogenic acid. In other aspects, the chlorogenic acid may be in a pure form. In other aspects, chlorogenic acid may be in the form of an extract. In some aspects, the chlorogenic acid may be in the derived from green coffee bean or coffee-based products. In some aspects the chlorogenic acid is in the form of green coffee bean extract.

In some aspects, the composition may be a feed composition for non-human animals.

In some aspects, the composition may be produced as an additive to feed for non-human animals.

In some aspects, the composition may be in pellet form to be added to non-human animal feed or as feed itself.

In some aspects, the composition may be in the form of a free choice supplement or a block.

In some aspects, the composition may be in the form of a liquid.

In some aspects, the composition may be administered in the form of a pill.

In some aspects, the composition may be administered in the form of a suppository.

In some aspects, the composition may be administered in the form of a bolus.

In some aspects, the composition may be administered in the form of an injection.

In some aspects, the composition is administered to the non-human animal following a stressor event. The stressor event could be in the form of, but is not limited to, an enterotoxigenic E. coli challenge, heat stress, abrupt diet changes, transportation, weaning, overcrowding, poor handling, feed restriction, nutritional deficiencies, exposure to pathogens, mycotoxins, sudden environmental changes, dysbiosis, bacterial toxins, chronic stress, social stress, off-feed events, vaccination, among other sources of animal stress.

In some aspects, the composition may be administered to the non-human animal at any time for general health benefits.

### I. Feed Composition

One aspect of the present disclosure encompasses feed composition for non-human animals. The non-human animal may be a ruminant or a non-ruminant animal. Examples of ruminant animals include but are not limited to cattle, wild bovines, goats, sheep, giraffes, deer, gazelles, or antelopes. Examples of non-ruminant animals include but are not limited to swine, poultry, horses, dogs, or cats.

In particular, non-human animals may be livestock or poultry.

The compositions comprise a basal animal diet supplemented with Bacillus and chlorogenic acid. The incorporation of Bacillus and chlorogenic acid into a feed composition for administration to an animal has surprisingly been found to be extremely effective in improving intestinal integrity, body weight gain, and lactation in said animal.

a. Bacillus

The amount of Bacillus in a feed composition can and will vary depending on the body weight, age, and medical condition of the non-human animal and can be determined experimentally. Generally, the amount of Bacillus present in the formulation will be an effective amount to improve characteristics in the non-human animal. The term “effective amount” describes an amount of Bacillus present in a feed composition sufficient to produce a noticeable effect, for example the improved weight gain following a stressor event, as determined according to observations described herein. The effective amount will depend on factors such as individual animal parameters including age, physical condition, size and weight; concurrent treatments; the frequency of treatment; or the mode of administration. These factors are well known to those of ordinary skill in the art.

The species of Bacillus may be any species that is included under the genus of Bacillus. The strain of Bacillus may be any strain that falls under the genus of Bacillus. The strain of Bacillus may be any strain that was previously under the genus of Bacillus or any strain that eventually is included under the genus of Bacillus. The species of Bacillus may include, but is not limited to, Bacillus subtilis, Bacillus velezensis, Bacillus amyloliquefaciens, Bacillus lichenformis, Bacillus pumilus, Bacillus mojavensis, Bacillus siamensis, Bacillus vallismortis, Bacillus sonorensis, Bacillus tequilensis, Bacillus paralicheniformis, Bacillus glycinifermentans, Bacillus megaterium, Bacillus aryabhattai, Bacillus altitudinis, Bacillus safensis, Bacillus halotolerans, Bacillus endophyticus, Bacillus simplex, Bacillus firmus, Bacillus toyonensis, Bacillus inaquosorum, Bacillus cabrialesii, Bacillus nakamurai, Bacillus coagulans, Bacillus clausii, Bacillus indicus, Bacillus subtilis var. natto, Bacillus lichenformis, Bacillus safensis, Bacillus glycinfermentans, Bacillus flexus, Bacillus mojavensis, Bacillus marisflavi, Bacillus altitudinis, Bacillus horneckiae, Bacillus safensis, Bacillus boroniphilus, Bacillus oceanisediminis, Bacillus kribbensis, Bacillus cereus var. toyoi, Bacillus coagulans GBI-30, 6086, Bacillus clausii UBBC-07/SIN/O/C/N/R, Bacillus indicus HU36, Bacillus toyonensis BCT-7112, Bacillus subtilis HU58, PXN-21, DSM 32315, Bacillus lichenformis DSM5749/5750, Bacillus altitudinis 41KF2b, Bacillus aryabhattai B8W22, Bacillus cabrialesii TE3^{T}, Bacillus sonorensis, Bacillus safensis FO-36b, Bacillus halotolerans, Bacillus siamensis, Bacillus endophyticus, Bacillus firmus, Bacillus megaterium, Bacillus korlensis, Bacillus niacin, Bacillus flexus, Bacillus marisflavi, Bacillus oceanisediminis, Bacillus lehensis, Bacillus thermoruber, Bacillus kribbensis, Bacillus boroniphilus, Bacillus alveayuensis, Weizmannia coagulans (formerly Bacillus coagulans), Priestia megaterium (formerly Bacillus megaterium), Priestia aryabhattai (formerly Bacillus aryabhattai), Alkalihalobacillus clausii (formerly Bacillus clausii), Alkalihalobacillus halodurans (formerly Bacillus halodurans), Lysinibacillus sphaericus (formerly Bacillus sphaericus), or a combination thereof. Preferably, the Bacillus species may be selected from the list comprising: Bacillus subtilis, Bacillus velezensis, Bacillus amyloliquefaciens, or a combination thereof. In some aspects, the strains of Bacillus may be selected from Bacillus amyloliquefaciens BA22 (BA22), Bacillus amyloliquefaciens BA1013 (BA1013), Bacillus velezensis ZBG17 (ZBG17), Bacillus subtilis CD1781 (CD1781 or also known as Bacillus velezensis NRRL B-67259), Bacillus velezensis CGS1.1 (CGS1.1), or a combination thereof.

The amount of Bacillus present in the feed may be at a feed concentration of about 1×10⁴-5×10¹⁰ CFU/head/day for ruminants and about 1×10⁴-5×10¹⁰ CFU/g of feed or equivalent amount supplemented via drinking water for monogastrics.

In some aspects, the amount of Bacillus present in a feed additive or supplement may be conditioned on the body weight, age, and medical condition of the non-human animal. The concentration of Bacillus would be an effective amount as defined above.

b. Chlorogenic Acid

The amount of chlorogenic acid in a feed composition can and will vary depending on the body weight, age, and medical condition of the non-human animal and can be determined experimentally. Generally, the amount of chlorogenic acid present in the formulation will be an effective amount to improve characteristics in the non-human animal. The term “effective amount” describes an amount of chlorogenic acid present in a feed composition sufficient to produce a noticeable effect, for example the improved weight gain following a stressor event, as determined according to observations described herein. The effective amount will depend on factors such as individual animal parameters including age, physical condition, size and weight; concurrent treatments; the frequency of treatment; or the mode of administration. These factors are well known to those of ordinary skill in the art.

The amount of chlorogenic acid present in the feed may be at a concentration of about 1 mg to 11 g/head/day for ruminants and 2-2,000 ppm of complete feed or equivalent amount delivered via drinking water for monogastrics.

In another nonlimiting aspect, the amount of chlorogenic acid present in a feed, feed additive, supplement is conditioned on the body weight, age, and medical condition of the non-human animal. The concentration of chlorogenic acid would be an effective amount as defined above.

Sources of chlorogenic acid include, but are not limited to, coffee beans, green coffee beans, coffee, green coffee, apples, artichoke, betel, burdock, carrots, eggplants, Eucommia, grapes, honeysuckle, kiwi fruit, pears, plums, potatoes, tea leaves, tobacco leaves, tomatoes, wormwood, tea leaves, zucchini, broccoli, onion, spinach, peas, green pepper, okra, cabbage, sage, rosemary, oregano, yams, chicory, rosemary, sweet potatoes, bay leaves, mustard, celery, collard greens, purple lettuce, arugula, chard, basil, asparagus, crisp lettuce, cilantro, escarole, okra, watercress, arracacha, extracts thereof, among others. Other potential sources of chlorogenic acid include any source that contains a detectable amount of chlorogenic acid, or any combination thereof.

In some aspects, the chlorogenic acid is derived from one source. In some aspects, the chlorogenic acid is derived from a combination of sources of chlorogenic acid. In another aspect, the chlorogenic acid is a purified form of chlorogenic acid. In another aspect, the chlorogenic acid is a derivative of chlorogenic acid.

c. Basal Animal Diet

A basal animal diet suitable for a feed composition of the disclosure can and will vary depending on the intended animal, the weight of the animal, and the stage of development of the animal among other variables.

The terms “feed”, “food”, and “feed formulation” are used herein interchangeably and may refer to any feed composition normally fed to an animal. Basal animal diets normally fed to an animal are known in the art. A basal animal diet may include one or more components of an animal feed. Non-limiting examples of feed matter or animal feed matter may include, without limitation: corn or a component of corn, such as, for example, corn meal, corn fiber, corn hulls, corn DDGS (distiller's dried grain with solubles), silage, ground corn, corn germ, corn gluten, corn oil, or any other portion of a corn plant; soy or a component of soy, such as, for example, soy oil, soy meal, soy hulls, soy silage, ground soy, or any other portion of a soy plant; wheat or any component of wheat, such as, for example, wheat meal, wheat fiber, wheat hulls, wheat chaff, ground wheat, wheat germ, or any other portion of a wheat plant; rice or any component of rice, such as, for example, rice meal, rice fiber, rice hulls, rice chaff, ground rice, rice germ, or any other portion of a rice plant; canola, such as, for example, canola oil, canola meal, canola protein, canola hulls, ground canola, or any other portion of a canola plant; sunflower or a component of a sunflower plant; sorghum or a component of a sorghum plant; sugar beet or a component of a sugar beet plant; cane sugar or a component of a sugarcane plant; barley or a component of a barley plant; palm oil, palm kernel or a component of a palm plant; glycerol; corn steep liquor; a waste stream from an agricultural processing facility; lecithin; rumen protected fats; molasses; soy molasses; flax; peanuts; peas; oats; grasses, such as orchard grass and fescue; fish meal, meat & bone meal; feather meal; and poultry byproduct meal; and alfalfa and/or clover used for silage or hay; and various combinations of any of the feed ingredients set forth herein, or other feed ingredients generally known in the art.

As it will be recognized in the art, a basal animal diet may further be supplemented with amino acids, vitamins, minerals, and other feed additives such as other types of enzymes, organic acids, essential oils, probiotics, prebiotics, antioxidants, pigments, anti-caking agents, and the like, as described further below. A basal animal diet may be formulated for administration to any animal subject. Animal subjects may be as described below.

The basal animal diets may optionally comprise at least one additional nutritive and/or pharmaceutical agent. For instance, the at least one additional nutritive and/or pharmaceutical agent may be selected from the group consisting of vitamin, mineral, amino acid, antioxidant, probiotic, essential fatty acid, and pharmaceutically acceptable excipient. The compositions may include one additional nutritive and/or pharmaceutical component or a combination of any of the foregoing additional components in varying amounts. Suitable examples of each additional component are detailed below.

i. Vitamins

Optionally, the animal feed formulation may include one or more vitamins. Suitable vitamins for use in the dietary supplement include vitamin C, vitamin A, vitamin E, vitamin B12, vitamin K, riboflavin, niacin, vitamin D, vitamin B6, folic acid, pyridoxine, thiamine, pantothenic acid, and biotin. The form of the vitamin may include salts of the vitamin, derivatives of the vitamin, compounds having the same or similar activity of a vitamin, and metabolites of a vitamin.

The animal feed formulation may include one or more forms of an effective amount of any of the vitamins described herein or otherwise known in the art. Exemplary vitamins include vitamin K, vitamin D, vitamin C, and biotin. An “effective amount” of a vitamin typically quantifies an amount at least about 10% of the United States Recommended Daily Allowance (“RDA”) of that particular vitamin for a subject. It is contemplated, however, that amounts of certain vitamins exceeding the RDA may be beneficial for certain animals. For example, the amount of a given vitamin may exceed the applicable RDA by 100%, 200%, 300%, 400%, 500% or more.

ii. Minerals

Generally, the animal feed formulation may include one or more minerals or mineral sources. Non-limiting examples of minerals include, without limitation, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

Generally speaking, the animal feed formulation may include one or more forms of an effective amount of any of the minerals described herein or otherwise known in the art. An “effective amount” of a mineral typically quantifies an amount at least about 10% of the United States Recommended Daily Allowance (“RDA”) of that particular mineral for a subject. It is contemplated, however, that amounts of certain minerals exceeding the RDA may be beneficial for certain subjects. For example, the amount of a given mineral may exceed the applicable RDA by 100%, 200%, 300%, 400%, 500% or more. Typically, the amount of mineral included in the dietary supplement may range from about 1 mg to about 1500 mg, about 5 mg to about 500 mg, or from about 50 mg to about 500 mg per dosage.

iii. Essential Fatty Acids

Optionally, the animal feed formulation may include a source of an essential fatty acid. The essential fatty acid may be isolated or it may be an oil source or fat source that contains an essential fatty acid. In some aspects, the essential fatty acid may be a polyunsaturated fatty acid (PUFA), which has at least two carbon-carbon double bonds generally in the cis-configuration. The PUFA may be a long chain fatty acid having at least 18 carbons atoms. The PUFA may be an omega-3 fatty acid in which the first double bond occurs in the third carbon-carbon bond from the methyl end of the carbon chain (i.e., opposite the carboxyl acid group). Examples of omega-3 fatty acids include alpha-linolenic acid (18:3, ALA), stearidonic acid (18:4), eicosatetraenoic acid (20:4), eicosapentaenoic acid (20:5; EPA), docosatetraenoic acid (22:4), n-3 docosapentaenoic acid (22:5; n-3DPA), and docosahexaenoic acid (22:6; DHA). The PUFA may also be an omega-5 fatty acid, in which the first double bond occurs in the fifth carbon-carbon bond from the methyl end. Exemplary omega-5 fatty acids include myristoleic acid (14:1), myristoleic acid esters, and cetyl myristoleate. The PUFA may also be an omega-6 fatty acid, in which the first double bond occurs in the sixth carbon-carbon bond from the methyl end. Examples of omega-6 fatty acids include linoleic acid (18:2), gamma-linolenic acid (18:3), eicosadienoic acid (20:2), dihomo-gamma-linolenic acid (20:3), arachidonic acid (20:4), docosadienoic acid (22:2), adrenic acid (22:4), and n-6 docosapentaenoic acid (22:5). The fatty acid may also be an omega-9 fatty acid, such as oleic acid (18:1), eicosenoic acid (20:1), mead acid (20:3), erucic acid (22:1), and nervonic acid (24:1).

In some aspects, the essential fatty acid source may be a seafood-derived oil. The seafood may be a vertebrate fish or a marine organism, such that the oil may be fish oil or marine oil. The long chain (20 C, 22 C) omega-3 and omega-6 fatty acids are found in seafood. The ratio of omega-3 to omega-6 fatty acids in seafood ranges from about 8:1 to 20:1. Seafood from which oil rich in omega-3 fatty acids may be derived include, but are not limited to, abalone scallops, albacore tuna, anchovies, catfish, clams, cod, gem fish, herring, lake trout, mackerel, menhaden, orange roughly, salmon, sardines, sea mullet, sea perch, shark, shrimp, squid, trout, and tuna.

In some aspects, the essential fatty acid source may be a plant-derived oil. Plant and vegetable oils are rich in omega-6 fatty acids. Some plant-derived oils, such as flaxseed oil, are especially rich in omega-3 fatty acids. Plant or vegetable oils are generally extracted from the seeds of a plant, but may also be extracted from other parts of the plant. Plant or vegetable oils that are commonly used for cooking or flavoring include, but are not limited to, acai oil, almond oil, amaranth oil, apricot seed oil, argan oil, avocado seed oil, babassu oil, ben oil, blackcurrant seed oil, Borneo tallow nut oil, borage seed oil, buffalo gourd oil, canola oil, carob pod oil, cashew oil, castor oil, coconut oil, coriander seed oil, corn oil, cottonseed oil, evening primrose oil, false flax oil, flax seed oil, grapeseed oil, hazelnut oil, hemp seed oil, kapok seed oil, lallemantia oil, linseed oil, macadamia oil, meadowfoam seed oil, mustard seed oil, okra seed oil, olive oil, palm oil, palm kernel oil, peanut oil, pecan oil, pequi oil, perilla seed oil, pine nut oil, pistachio oil, poppy seed oil, prune kernel oil, pumpkin seed oil, quinoa oil, ramtil oil, rice bran oil, safflower oil, sesame oil, soybean oil, sunflower oil, tea oil, thistle oil, walnut oil, or wheat germ oil. The plant-derived oil may also be hydrogenated or partially hydrogenated.

In some aspects, the essential fatty acid source may be an algae-derived oil. Commercially available algae-derived oils include those from Crypthecodinium cohnii and Schizochytrium sp. Other suitable species of algae, from which oil is extracted, include Aphanizomenon flos-aquae, Bacilliarophy sp., Botryococcus braunii, Chlorophyceae sp., Dunaliella tertiolecta, Euglena gracilis, Isochrysis galbana, Nannochloropsis salina, Nannochloris sp., Neochloris oleoabundans, Phaeodactylum tricornutum, Pleurochrysis carterae, Prymnesium parvum, Scenedesmus dimorphus, Spirulina sp., and Tetraselmis chui.

iv. Amino Acids

The animal feed formulation may optionally include from one to several amino acids. Suitable amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine or their hydroxy analogs. In some aspects, the amino acid will be selected from the essential amino acids. An essential amino acid is generally described as one that cannot be synthesized de novo by the organism, and therefore, must be provided in the diet. By way of non-limiting example, the essential amino acids for humans include: L-histidine, L-isoleucine, L-leucine, L-lysine, L-methionine, L-phenylalanine, L-valine and L-threonine.

v. Antioxidants

The animal feed formulation may include one or more suitable antioxidants. As will be appreciated by a skilled artisan, the suitability of a given antioxidant will vary depending upon the species to which the dietary supplement will be administered. Non-limiting examples of antioxidants include ascorbic acid and its salts, ascorbyl palmitate, ascorbyl stearate, anoxomer, N-acetylcysteine, benzyl isothiocyanate, o-, m- or p-amino benzoic acid (o is anthranilic acid, p is PABA), butylated hydroxyanisole (BHA), butylated hydroxytoluene (BHT), caffeic acid, canthaxantin, alpha-carotene, beta-carotene, beta-carotene, beta-apo-carotenoic acid, carnosol, carvacrol, catechins, cetyl gallate, chlorogenic acid, citric acid and its salts, p-coumaric acid, curcurin, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eugenol, ferulic acid, flavonoids, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, propyl gallate, polyphosphates, quercetin, trans-resveratrol, rosmarinic acid, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., Ionox 100), 2,4-(tris-3′,5′-bi-tert-butyl-4′-hydroxybenzyl)-mesitylene (i.e., Ionox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, zeaxanthin, or combinations thereof.

Natural antioxidants that may be included in the dietary supplement include, but are not limited to, apple peel extract, blueberry extract, carrot juice powder, clove extract, coffee berry, coffee bean extract, cranberry extract, eucalyptus extract, ginger powder, grape seed extract, green tea, olive leaf, parsley extract, peppermint, pimento extract, pomace, pomegranate extract, rice bran extract, rosehips, rosemary extract, sage extract, tart cherry extract, tomato extract, turmeric, and wheat germ oil.

vi. Anti-Inflammatory Agents

The animal feed formulation may optionally include at least one anti-inflammatory agent. In some aspects, the anti-inflammatory agent may be a synthetic non-steroidal anti-inflammatory drug (NSAID) such as acetylsalicylic acid, dichlophenac, indomethacin, oxamethacin, ibuprofen, indoprofen, naproxen, ketoprofen, mefenamic acid, metamizole, piroxicam, and celecoxib. In other aspects, the anti-inflammatory agent may be a prohormone that modulates inflammatory processes. Suitable prohormones having this property include prohormone convertase 1, proopiomelanocortin, prohormone B-type natriuretic peptide, SMR1 prohormone, and the like. In another aspect, the anti-inflammatory agent may be an enzyme having anti-inflammatory effects. Examples of anti-inflammatory enzymes include bromelain, papain, serrapeptidase, and proteolytic enzymes such as pancreatin (a mixture of trypsin, amylase and lipase).

In some aspects, the anti-inflammatory agent may be a peptide with anti-inflammatory effects. For example, the peptide may be an inhibitor of phospholipase A2, such as antiflammin-1, a peptide that corresponds to amino acid residues 246-254 of lipocortin; antiflammin-2, a peptide that corresponds to amino acid residues 39-47 of uteroglobin; S7 peptide, which inhibits the interaction between interleukin 6 and interleukin 6 receptor; RP1, a prenyl protein inhibitor; and similar peptides. Alternatively, the anti-inflammatory peptide may be cortistatin, a cyclic neuropeptide related to somatostatin, or peptides that correspond to an N-terminal fragment of SV-IV protein, a conserved region of E-, L-, and P-selectins, and the like. Other suitable anti-inflammatory preparations include collagen hydrolysates and milk micronutrient concentrates (e.g., MicroLactin® available from Stolle Milk Biologics, Inc., Cincinnati, OH), as well as milk protein hydrolysates, casein hydrolysates, whey protein hydrolysates, and plant protein hydrolysates.

In some aspects, the anti-inflammatory agent may be a probiotic that has been shown to modulate inflammation. Suitable immunomodulatory probiotics include lactic acid bacteria such as acidophilli, lactobacilli, and bifidophilli. In some aspects, the anti-inflammatory agent may be a plant extract having anti-inflammatory properties. Non-limiting examples of suitable plant extracts with anti-inflammatory benefits include blueberries, boswella, black catechu and Chinese skullcap, celery seed, chamomile, cherries, devil's claw, eucalyptus, evening primrose, ginger, hawthorne berries, horsetail, Kalopanax pictus bark, licorice root, turmeric, white wallow, willow bark, and yucca.

vii. Herbals

The animal feed formulation may optionally include at least one herb or herbal derivative. Suitable herbals and herbal derivatives, as used herein, refer to herbal extracts, and substances derived from plants and plant parts, such as leaves, flowers, and roots, without limitation. Non-limiting exemplary herbals and herbal derivatives include agrimony, alfalfa, aloe vera, amaranth, angelica, anise, barberry, basil, bayberry, bee pollen, birch, bistort, blackberry, black cohosh, black walnut, blessed thistle, blue cohosh, blue vervain, boneset, borage, buchu, buckthorn, bugleweed, burdock, capsicum, cayenne, caraway, cascara sagrada, catnip, celery, centaury, chamomile, chaparral, chickweed, chicory, chinchona, cloves, coltsfoot, comfrey, cornsilk, couch grass, cramp bark, culver's root, cyani, cornflower, damiana, dandelion, devils claw, dong quai, echinacea, elecampane, ephedra, eucalyptus, evening primrose, eyebright, false unicorn, fennel, fenugreek, figwort, flaxseed, garlic, gentian, ginger, ginseng, golden seal, gotu kola, gum weed, hawthorn, hops, horehound, horseradish, horsetail, hoshouwu, hydrangea, hyssop, iceland moss, irish moss, jojoba, juniper, kelp, lady's slipper, lemon grass, licorice, lobelia, mandrake, marigold, marjoram, marshmallow, mistletoe, mullein, mustard, myrrh, nettle, oatstraw, oregon grape, papaya, parsley, passion flower, peach, pennyroyal, peppermint, periwinkle, plantain, pleurisy root, pokeweed, prickly ash, psyllium, quassia, queen of the meadow, red clover, red raspberry, redmond clay, rhubarb, rose hips, rosemary, rue, safflower, saffron, sage, St. John's wort, sarsaparilla, sassafras, saw palmetto, skullcap, senega, senna, shepherd's purse, slippery elm, spearmint, spikenard, squawvine, stillingia, strawberry, taheebo, thyme, uva ursi, valerian, violet, watercress, white oak bark, white pine bark, wild cherry, wild lettuce, wild yam, willow, wintergreen, witch hazel, wood betony, wormwood, yarrow, yellow dock, yerba santa, yucca and combinations thereof.

viii. Pigments

The animal feed formulation may optionally include at least one pigment. Suitable non-limiting pigments include actinioerythrin, alizarin, alloxanthin, β-apo-2′-carotenal, apo-2-lycopenal, apo-6′-lycopenal, astacein, astaxanthin, azafrinaldehyde, aacterioruberin, aixin, α-carotine, β-carotine, 7-carotine, 0-carotenone, canthaxanthin, capsanthin, capsorubin, citranaxanthin, citroxanthin, crocetin, crocetinsemialdehyde, crocin, crustaxanthin, cryptocapsin, α-cryptoxanthin, β-cryptoxanthin, cryptomonaxanthin, cynthiaxanthin, decaprenoxanthin, dehydroadonirubin, diadinoxanthin, 1,4-diamino-2,3-dihydroanthraquinone, 1,4-dihydroxyanthraquinone, 2,2′-diketospirilloxanthin, eschscholtzxanthin, eschscholtzxanthone, flexixanthin, foliachrome, fucoxanthin, gazaniaxanthin, hexahydrolycopene, hopkinsiaxanthin, hydroxyspheriodenone, isofucoxanthin, loroxanthin, lutein, luteoxanthin, lycopene, lycopersene, lycoxanthin, morindone, mutatoxanthin, neochrome, neoxanthin, nonaprenoxanthin, OH-Chlorobactene, okenone, oscillaxanthin, paracentrone, pectenolone, pectenoxanthin, peridinin, phleixanthophyll, phoeniconone, phoenicopterone, phoenicoxanthin, physalien, phytofluene, pyrrhoxanthininol, quinones, rhodopin, rhodopinal, rhodopinol, rhodovibrin, rhodoxanthin, rubixanthone, saproxanthin, semi-α-carotenone, semi-β-carotenone, sintaxanthin, siphonaxanthin, siphonein, spheroidene, tangeraxanthin, torularhodin, torularhodin methyl ester, torularhodinaldehyde, torulene, 1,2,4-trihydroxyanthraquinone, triphasiaxanthin, trollichrome, vaucheriaxanthin, violaxanthin, wamingone, xanthin, zeaxanthin, α-zeacarotene, or combinations thereof.

ix. Pharmaceutical Acceptable Agents

The animal feed formulation may optionally include at least one pharmaceutical acceptable agent. Suitable non-limiting pharmaceutically acceptable agents include an acid/alkaline-labile drug, a pH dependent drug, or a drug that is a weak acid or a weak base. Examples of acid-labile drugs include statins (e.g., pravastatin, fluvastatin and atorvastatin), antibiotics (e.g., penicillin G, ampicillin, streptomycin, erythromycin, clarithromycin and azithromycin), nucleoside analogs (e.g., dideoxyinosine (ddI or didanosine), dideoxyadenosine (ddA), dideoxycytosine (ddC), salicylates (e.g., aspirin), digoxin, bupropion, pancreatin, midazolam, and methadone. Drugs that are only soluble at acid pH include nifedipine, emonapride, nicardipine, amosulalol, noscapine, propafenone, quinine, dipyridamole, josamycin, dilevalol, labetalol, enisoprost, and metronidazole. Drugs that are weak acids include phenobarbital, phenytoin, zidovudine (AZT), salicylates (e.g., aspirin), propionic acid compounds (e.g., ibuprofen), indole derivatives (e.g., indomethacin), fenamate compounds (e.g., meclofenamic acid), pyrrolealkanoic acid compounds (e.g., tolmetin), cephalosporins (e.g., cephalothin, cephalaxin, cefazolin, cephradine, cephapirin, cefamandole, and cefoxitin), 6-fluoroquinolones, and prostaglandins. Drugs that are weak bases include adrenergic agents (e.g., ephedrine, desoxyephedrine, phenylephrine, epinephrine, salbutamol, and terbutaline), cholinergic agents (e.g., physostigmine and neostigmine), antispasmodic agents (e.g., atropine, methantheline, and papaverine), curariform agents (e.g., chlorisondamine), tranquilizers and muscle relaxants (e.g., fluphenazine, thioridazine, trifluoperazine, chlorpromazine, and triflupromazine), antidepressants (e.g., amitriptyline and nortriptyline), antihistamines (e.g., diphenhydramine, chlorpheniramine, dimenhydrinate, tripelennamine, perphenazine, chlorprophenazine, and chlorprophenpyridamine), cardioactive agents (e.g., verapamil, diltiazem, gallapomil, cinnarizine, propranolol, metoprolol and nadolol), antimalarials (e.g., chloroquine), analgesics (e.g., propoxyphene and meperidine), antifungal agents (e.g., ketoconazole and itraconazole), antimicrobial agents (e.g., cefpodoxime, proxetil, and enoxacin), caffeine, theophylline, and morphine. In some aspects, the drug may be a biphosphonate or another drug used to treat osteoporosis. Non-limiting examples of a biphosphonate include alendronate, ibandronate, risedronate, zoledronate, pamidronate, neridronate, olpadronate, etidronate, clodronate, and tiludronate. Other suitable drugs include estrogen, selective estrogen receptor modulators (SERMs), and parathyroid hormone (PTH) drugs. In yet another aspect, the drug may be an antibacterial agent. Suitable antibiotics include aminoglycosides (e.g., amikacin, gentamicin, kanamycin, neomycin, netilmicin, streptomycin, and tobramycin), carbecephems (e.g., loracarbef), a carbapenem (e.g., certapenem, imipenem, and meropenem), cephalosporins (e.g., cefadroxil cefazolin, cephalexin, cefaclor, cefamandole, cephalexin, cefoxitin, cefprozil, cefuroxime, cefixime, cefdinir, cefditoren, cefoperazone, cefotaxime, cefpodoxime, ceftazidime, ceftibuten, ceftizoxime, and ceftriaxone), macrolides (e.g., azithromycin, clarithromycin, dirithromycin, erythromycin, and troleandomycin), monobactam, penicillins (e.g., amoxicillin, ampicillin, carbenicillin, cloxacillin, dicloxacillin, nafcillin, oxacillin, penicillin G, penicillin V, piperacillin, and ticarcillin), polypeptides (e.g., bacitracin, colistin, and polymyxin B), quinolones (e.g., ciprofloxacin, enoxacin, gatifloxacin, levofloxacin, lomefloxacin, moxifloxacin, norfloxacin, ofloxacin, and trovafloxacin), sulfonamides (e.g., mafenide, sulfacetamide, sulfamethizole, sulfasalazine, sulfisoxazole, and trimethoprim-sulfamethoxazole), and tetracyclines (e.g., demeclocycline, doxycycline, minocycline, and oxytetracycline). In some aspects, the drug may be an antiviral protease inhibitor (e.g., amprenavir, fosamprenavir, indinavir, lopinavir/ritonavir, ritonavir, saquinavir, and nelfinavir). In still another aspect, the drug may be a cardiovascular drug. Examples of suitable cardiovascular agents include cardiotonic agents (e.g., digitalis (digoxin), ubidecarenone, and dopamine), vasodilating agents (e.g., nitroglycerin, captopril, dihydralazine, diltiazem, and isosorbide dinitrate), antihypertensive agents (e.g., alpha-methyldopa, chlortalidone, reserpine, syrosingopine, rescinnamine, prazosin, phentolamine, felodipine, propanolol, pindolol, labetalol, clonidine, captopril, enalapril, and lisonopril), beta blockers (e.g., levobunolol, pindolol, timolol maleate, bisoprolol, carvedilol, and butoxamine), alpha blockers (e.g., doxazosin, prazosin, phenoxybenzamine, phentolamine, tamsulosin, alfuzosin, and terazosin), calcium channel blockers (e.g., amlodipine, felodipine, nicardipine, nifedipine, nimodipine, nisoldipine, nitrendipine, lacidipine, lercanidipine, verapamil, gallopamil, and diltiazem), monensin, avilamycin, salinomycin, narasin, diclaserol, tylosin, bacitracin, bacitracin zinc, and anticlot agents (e.g., dipyrimadole).

x. Excipients

A variety of commonly used excipients in animal feed formulation may be selected on the basis of compatibility with the active ingredients. Non-limiting examples of suitable excipients include an agent selected from the group consisting of non-effervescent disintegrants, a coloring agent, a flavor-modifying agent, an oral dispersing agent, a stabilizer, a preservative, a diluent, a compaction agent, a lubricant, a filler, a binder, taste masking agents, an effervescent disintegration agent, and combinations of any of these agents.

In some aspects, the excipient may be a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof. The polypeptide may be any arrangement of amino acids ranging from about 100 to about 300,000 daltons.

In some aspects, the excipient may be a filler. Suitable fillers include carbohydrates, inorganic compounds, and polyvinylpirrolydone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and sorbitol.

The excipient may comprise a non-effervescent disintegrant. Suitable examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth.

In some aspects, the excipient may be an effervescent disintegrant. By way of non-limiting example, suitable effervescent disintegrants include sodium bicarbonate in combination with citric acid and sodium bicarbonate in combination with tartaric acid.

The excipient may comprise a preservative. Suitable examples of preservatives include antioxidants, such as a-tocopherol or ascorbate, and antimicrobials, such as parabens, chlorobutanol or phenol.

In some aspects, the excipient may include a diluent. Diluents suitable for use include pharmaceutically acceptable saccharides such as sucrose, dextrose, lactose, microcrystalline cellulose, fructose, xylitol, and sorbitol; polyhydric alcohols; a starch; pre-manufactured direct compression diluents; and mixtures of any of the foregoing.

The excipient may include flavors. Flavors incorporated into the outer layer may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oil, such as lemon oil, orange oil, grape and grapefruit oil, fruit essences including apple, peach, pear, strawberry, raspberry, cherry, plum, pineapple, and apricot.

In some aspects, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, sylitol, and the like.

In some aspects, the excipient may be a lubricant. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

The excipient may be a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some aspects, it may be desirable to provide a coloring agent in the outer layer. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in the present disclosure.

The excipient may include a taste-masking agent. Taste-masking materials include, e.g., cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other aspects, additional taste-masking materials contemplated are those described in U.S. Pat. Nos. 4,851,226; 5,075,114; and 5,876,759, each of which is hereby incorporated by reference in its entirety.

In some aspects, the excipient may include a pH modifier. In certain aspects, the pH modifier may include sodium carbonate or sodium bicarbonate.

The amount and types of ingredients and other excipients useful in the animal feed formulation are described further in the examples.

### II. Feed Additive Composition

One aspect of the present disclosure encompasses feed additive compositions for non-human animals comprising Bacillus and chlorogenic acid. Other optional additives may be further included. The feed additive composition may be added to a basal animal diet for administration to the non-human animals. The feed additive composition may be formulated with basal animal diets to prepare the feed compositions described in Section I.

In some aspects, the range of Bacillus is 1×10⁴-5×10¹⁰ CFU/head/day and chlorogenic acid 5×10⁻⁵ to 11 g/head/day for ruminants, and Bacillus supplied at 1×10⁴-5×10¹⁰ CFU/g of feed and chlorogenic acid supplied at 2-2,000 ppm of feed for monogastrics.

In some aspects, a feed additive composition may be introduced to a basal animal diet by way of various methods, depending on whether the feed additive composition is in a liquid or solid form. Non-limiting examples of introducing the feed additive composition to a basal animal diet may be formulating the feed additive composition into the basal animal diet, top-dressing the solid composition of a basal animal diet, spraying a liquid feed additive composition onto a basal animal diet, or combinations thereof. It will be recognized that, when the feed additive is introduced to a basal animal diet, the amount of the feed additive introduced to a basal animal diet is sufficient to provide the therapeutically effective amount of the Bacillus and chlorogenic acid in the diet of the animal.

In addition to the Bacillus and chlorogenic acid, a feed additive composition may further comprise at least one additional ingredient such as vitamins, minerals, amino acids, antioxidants, probiotics, essential fatty acids, and pharmaceutically acceptable excipients. Such ingredients may be as described in Section I(c) above.

### III. Methods of Using

Another aspect of the disclosure encompasses methods of using a feed composition or feed additive composition. The methods comprise administering the animal feed composition or feed additive composition to non-human animals, in particular poultry and cattle. Preferably, a feed composition or feed additive composition is administered orally to non-human animals. A feed composition may be as described in Section (I) or a feed additive composition may be as described in Section (II). In some aspects the feed composition or feed additive composition may be administered following a stressor event to alleviate and/or treat negative effects of said stressor event.

The timing and duration of administration of a composition of the disclosure to an animal can and will vary. The feed composition or feed additive composition may be administered throughout the period of feeding the animal. Alternatively, the feed composition or feed additive composition may be administered at specific periods during the growth and development of the animal, for instance, during periods of lactation. A composition may also be administered at various intervals. For instance, a composition may be administered daily, weekly, monthly, or over a number of months. In some aspects, a composition is administered weekly. In some aspects, a composition is administered monthly. Preferably, a composition is administered daily. As it will be recognized in the art, the duration of treatment can and will vary depending on the growth and health of the animal.

The timing following the stressor event for administration of the feed composition or feed additive composition can and will vary depending on the animal and the type of stressor event. The first administration of the feed composition or feed additive composition may immediately follow the stressor event to alleviate and/or treat negative effects of said stressor event. The first administration of the feed composition or feed additive composition may be within the first day following the stressor event to alleviate and/or treat negative effects of said stressor event. The first administration of the feed composition or feed additive composition may be within the first two days following the stressor event to alleviate and/or treat negative effects of said stressor event. The first administration of the feed composition or feed additive composition may be within the first three days following the stressor event to alleviate and/or treat negative effects of said stressor event. The first administration of the feed composition or feed additive composition may be within the first four days following the stressor event to alleviate and/or treat negative effects of said stressor event. The first administration of the feed composition or feed additive composition may be within the first five days following the stressor event to alleviate and/or treat negative effects of said stressor event. The first administration of the feed composition or feed additive composition may be within the first six days following the stressor event to alleviate and/or treat negative effects of said stressor event. The first administration of the feed composition or feed additive composition may be within the first week following the stressor event to alleviate and/or treat negative effects of said stressor event. The first administration of the feed composition or feed additive composition may be within the first two weeks following the stressor event to alleviate and/or treat negative effects of said stressor event. The first administration of the feed composition or feed additive composition may be within the first three weeks following the stressor event to alleviate and/or treat negative effects of said stressor event. The first administration of the feed composition or feed additive composition may be within the first month following the stressor event to alleviate and/or treat negative effects of said stressor event. The first administration of the feed composition or feed additive composition may be within the first two months following the stressor event to alleviate and/or treat negative effects of said stressor event. The first administration of the feed composition or feed additive composition may be within the first three months following the stressor event to alleviate and/or treat negative effects of said stressor event. The first administration of the feed composition or feed additive composition may be within the first four months following the stressor event to alleviate and/or treat negative effects of said stressor event.

### Improving Overall Health

In some aspects, the present disclosure is directed to a method for improving weight gain in a non-human animal. The method comprises feeding the non-human animal the feed composition described in Section I or the feed additive composition in Section II. In some aspects, the present disclosure is directed to a method for improving weight gain in a non-human animal following a stressor event. In some aspects, the method comprises feeding the non-human animal the feed composition described in Section I or the feed additive composition in Section II following a stressor event. In some aspects, the stressor event may be any stressor event described herein.

In some aspects, the present disclosure is directed to a method for improving growth performance in a non-human animal. The method comprises feeding the non-human animal the feed composition described in Section I or the feed additive composition in Section II. In some aspects, the present disclosure is directed to a method for improving growth performance in a non-human animal following a stressor event. The method comprises feeding the non-human animal the feed composition described in Section I or the feed additive composition in Section II following a stressor event. In some aspects, the stressor event may be any stressor event described herein.

In some aspects, the present disclosure is directed to a method for improving the percentage of non-human animals that survive from the start to end of a defined period. The method comprises feeding the non-human animal the feed composition described in Section I or the feed additive composition in Section II. In some aspects, the present disclosure is directed to a method for improving the percentage of non-human animals that survive from the start to end of a defined period following a stressor event. The method comprises feeding the non-human animal the feed composition described in Section I or the feed additive composition in Section II following a stressor event. In some aspects, the stressor event may be any stressor event described herein.

In some aspects, the present disclosure is directed to a method for increasing weight gain following a stressor event in a non-human animal. The method comprises feeding the non-human animal the feed composition described in Section I or the feed additive composition in Section II. In some aspects, the present disclosure is directed to a method for increasing weight gain following a stressor event in a non-human animal following a stressor event. The method comprises feeding the non-human animal the feed composition described in Section I or the feed additive composition in Section II following a stressor event. In some aspects, the stressor event may be any stressor event described herein.

In some aspects, the present disclosure is directed to a method for decreasing diarrhea in a non-human animal. The method comprises feeding the non-human animal the feed composition described in Section I or the feed additive composition in Section II. In some aspects, the present disclosure is directed to a method for decreasing diarrhea in a non-human animal following a stressor event. The method comprises feeding the non-human animal the feed composition described in Section I or the feed additive composition in Section II following a stressor event. In some aspects, the stressor event may be any stressor event described herein.

In some aspects, the present disclosure is directed to a method for improving plasma lipopolysaccharide binding protein concentrations in a non-human animal. The method comprises feeding the non-human animal the feed composition described in Section I or the feed additive composition in Section II. In some aspects, the present disclosure is directed to a method for improving plasma lipopolysaccharide binding protein concentrations in a non-human animal following a stressor event. The method comprises feeding the non-human animal the feed composition described in Section I or the feed additive composition in Section II following a stressor event. In some aspects, the stressor event may be any stressor event described herein.

In some aspects, the present disclosure is directed to a method for improving plasma lipopolysaccharide binding protein concentrations following a stressor event in a non-human animal. The method comprises feeding the non-human animal the feed composition described in Section I or the feed additive composition in Section II. In some aspects, the present disclosure is directed to a method for improving plasma lipopolysaccharide binding protein concentrations following a stressor event in a non-human animal following a stressor event. The method comprises feeding the non-human animal the feed composition described in Section I or the feed additive composition in Section II following a stressor event. In some aspects, the stressor event may be any stressor event described herein.

In some aspects, the present disclosure is directed to a method for improving intestinal integrity in a non-human animal. The method comprises feeding the non-human animal the feed composition described in Section I or the feed additive composition in Section II. In some aspects, the present disclosure is directed to a method for improving intestinal integrity in a non-human animal following a stressor event. The method comprises feeding the non-human animal the feed composition described in Section I or the feed additive composition in Section II following a stressor event. In some aspects, the stressor event may be any stressor event described herein.

In some aspects, the present disclosure is directed to a method for improving gene expression of ileal tight junction protein including occluding, claudin-3, and tight junction protein-1, among others in a non-human animal. The method comprises feeding the non-human animal the feed composition described in Section I or the feed additive composition in Section II. In some aspects, the present disclosure is directed to a method for improving gene expression of ileal tight junction protein including occluding, claudin-3, and tight junction protein-1, among others in a non-human animal following a stressor event. The method comprises feeding the non-human animal the feed composition described in Section I or the feed additive composition in Section II following a stressor event. In some aspects, the stressor event may be any stressor event described herein.

In some aspects, the present disclosure is directed to a method for improving intestinal integrity in a non-human animal. The method comprises feeding the non-human animal the feed composition described in Section I or the feed additive composition in Section II. In some aspects, the present disclosure is directed to a method for improving intestinal integrity in a non-human animal following a stressor event. The method comprises feeding the non-human animal the feed composition described in Section I or the feed additive composition in Section II following a stressor event. In some aspects, the stressor event may be any stressor event described herein.

In some aspects, the present disclosure is directed to a method for improving milk quality in a non-human animal. The method comprises feeding the non-human animal the feed composition described in Section I or the feed additive composition in Section II. In some aspects, the present disclosure is directed to a method for improving milk quality in a non-human animal following a stressor event. The method comprises feeding the non-human animal the feed composition described in Section I or the feed additive composition in Section II following a stressor event. In some aspects, the stressor event may be any stressor event described herein.

In some aspects, the present disclosure is directed to a method for improving milk yield in a non-human animal. The method comprises feeding the non-human animal the feed composition described in Section I or the feed additive composition in Section II. In some aspects, the present disclosure is directed to a method for improving milk yield in a non-human animal following a stressor event. The method comprises feeding the non-human animal the feed composition described in Section I or the feed additive composition in Section II following a stressor event. In some aspects, the stressor event may be any stressor event described herein.

In some aspects, the present disclosure is directed to a method for improving milk protein concentration in a non-human animal. The method comprises feeding the non-human animal the feed composition described in Section I or the feed additive composition in Section II. In some aspects, the present disclosure is directed to a method for improving milk protein concentration in a non-human animal following a stressor event. The method comprises feeding the non-human animal the feed composition described in Section I or the feed additive composition in Section II following a stressor event. In some aspects, the stressor event may be any stressor event described herein.

### DEFINITIONS

Unless otherwise defined, all technical and scientific terms used herein have the same meaning as is commonly understood by one of skill in the art to which this disclosure belongs at the time of filing. If specifically defined, then the definition provided herein takes precedent over any dictionary or extrinsic definition. Further, unless otherwise required by context, singular terms shall include pluralities, and plural terms shall include the singular. Herein, the use of “or” means “and/or” unless stated otherwise. All patents and publications referred to herein are incorporated by reference.

When introducing elements of the present disclosure or the aspects thereof, the articles “a,” “an,” “the,” and “said” are intended to mean that there are one or more of the elements. The terms “comprising,” “including,” and “having” are intended to be inclusive and mean that there may be additional elements other than the listed elements.

Although the disclosure described herein is susceptible to various modifications and alternative iterations, specific aspects thereof have been described in greater detail above. It should be understood, however, that the detailed description is not intended to limit the disclosure to the specific aspects disclosed. Rather, it should be understood that the disclosure is intended to cover all modifications, equivalents, and alternatives falling within the spirit and scope of the disclosure as defined by the claim language.

As used herein, “administering” is used in its broadest sense to mean contacting a subject with a composition disclosed herein.

As used herein, the term “Bacillus” includes all species under the genus of Bacillus and all gram-positive, rod-shaped bacteria. The term includes the species: Bacillus amyloliquefaciens, Bacillus subtilis, Bacillus velezensis, as well as all other species included in the Bacillus genus. The term also includes all future reclassifications of the species currently or previously considered to be within the genus of Bacillus.

As used herein, the term “Bacillus amyloliquefaciens” means that gram-positive bacteria lineage Bacillus amyloliquefaciens. The term includes all strains of Bacillus amyloliquefaciens, including, but not limited to, BA22 and BA1013. The term also includes all future reclassifications of the strain currently or previously considered to be within the definition of Bacillus amyloliquefaciens.

As used herein, the term “Bacillus subtilis” means the gram-positive bacteria lineage Bacillus subtilis. The term includes all strains of Bacillus subtilis, including, but not limited to, CD1781 (also known as Bacillus velezensis NRRL B-67259). The term also includes all future reclassifications of the strain currently or previously considered to be within the definition of Bacillus subtilis.

As used herein, the term “Bacillus velezensis” means the gram-positive bacteria lineage Bacillus velezensis. The term includes all strains of Bacillus velezensis, including, but not limited to, ZVG17 and CGS1.1. The term also includes all future reclassifications of the strain currently or previously considered to be within the definition of Bacillus velezensis.

As used herein, the term “chlorogenic acid” means the polyphenol compound also known as coffee tannic acid, 3-caffeoylquinic acid, chlorogenate, heriguard, (1S,3R,4R,5R)-3-[(E)-3-(3,4-dihydroxyphenyl)prop-2-enoyl]oxy-1,4,5-trihydroxycyclohexane-1-carboxylic acid. The term chlorogenic acid also includes any source of chlorogenic acid. The term chlorogenic acid may also include any derivative of chlorogenic acid.

As used herein, the term “health events” during lactation can include, but are not limited to, metabolic disorders including hypocalcemia, ketosis, retained placenta, displaced abomasum among others.

As used herein, the term “improved milk yield” can include, but is not limited to, increased total milk production, increased energy-corrected milk production, increased frequency of milkings, increased duration of milking, increases in milk components, increased milk protein concentration, improved nutrition in the milk, among other improvements in milk quality and production.

As used herein, the term “improved overall health” means, but is not limited to, decrease in diarrhea, increase in weight gain, increased weight gain following a stressor event, improve plasma lipopolysaccharide binding protein concentrations, improve plasma lipopolysaccharide binding protein concentrations following a stressor event, improved intestinal integrity, improved gene expression of ileal tight junction proteins including occluding, claudin-3, tight junction protein-1, among others.

As used herein, the term “improvements in intestinal integrity” can include, improvement in tight junction protein function, decrease of lipopolysaccharide entering the bloodstream through the intestine, improvement in barrier function of the intestinal walls, recovery or increase in gene expression of ileal tight junction proteins such as Occludin, Claudin-3, Tight-junction 1, among others, decrease in large molecules entering the bloodstream through the intestinal barrier, improvement in intestinal barrier function as measured by transepithelial electrical resistance, increase in beneficial nutrient absorption, decrease the frequency and severity of negative health events caused by intestinal misfunction, among others.

As used herein, the term “non-human animal” means, but is not limited to, cattle, bull, pig, sheep, ram, chicken, turkey, goat, alpaca, horse, duck, donkey, goose, llama, domestic water buffalo, camel, deer, rabbit, dogs, cats, zebu, yak, gayal, reindeer, fowl, guineafowl, quail, helmeted guineafowl, Swedish flower hen, Indian game hen, Barbu d'Uccle, as well as other animals. The non-human animal may be a ruminant or a non-ruminant animal. Examples of ruminant animals include but are not limited to cattle, wild bovines, goats, sheep, giraffes, deer, gazelles, or antelopes. Examples of non-ruminant animals include but are not limited to swine, poultry, horses, dogs, or cats.

As used herein, the term “stressors” include, but are not limited to, enterotoxigenic E. coli challenge, heat stress, abrupt diet changes, transportation, weaning, overcrowding, poor handling, feed restriction, nutritional deficiencies, exposure to pathogens, mycotoxins, sudden environmental changes, dysbiosis, bacterial toxins, chronic stress, social stress, off-feed events, among other sources of animal stress.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the disclosure is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. A 24-Hour Feed Restriction Event Impacts Tight Junction Proteins and Physiological Markers of Inflammation. A Combination of 2 Bacillus amyloliquefaciens Strains Fed at 500,000 CFU/g Partially Alleviated the Negative Impact of the Feed Restriction Stress Event

### Experimental Design & Procedure:

The impact of Bacillus amyloliquefaciens on tight junction proteins and inflammation following a stressor event were evaluated. A total of 75 weanling barrows were utilized in this study. Pigs were weighed upon arrival to the facility and housed in 30 pens with 2-3 pigs/pen. Each pen was randomly assigned to one of 5 dietary treatments in a completely randomized design (Table 1).

TABLE 1
Dietary treatments

| Treatment | | Total Cell Count |
|---|---|---|
| Number | Treatments | CFU/g of feed |
| 1 | No Feed Restriction-Positive Control | |
| 2 | 24 hr Feed Restriction-Negative Control | |
| 3 | BA22 + BA1013 (0.5 × dose) | 5 × 10⁵ |
| 4 | BA22 + BA1013 (1 × dose) | 1 × 10⁶ |
| 5 | BA22 + BA1013 (2 × dose) | 2 × 10⁶ |

This experiment was conducted for 22 days. Pigs were weaned and arrived at the facility at 26-28 days of age. Upon arrival, pigs were weighed and allotted to one of 30 pens. All pigs were fed the same basal diet balanced to meet requirements (Table 2). Treatment 3-5 listed in Table 1 were mixed with the nursery diet (Table 2) that was offered to the pigs ad libitum upon arrival. In addition to the feed, pigs were offered ad libitum access to water throughout the trial. After 18 days of receiving treatment diets, feed was removed for a 24-hour period from the pens for pigs assigned to treatments 2-5. This created an off-feed event to simulate stress that occurs naturally in production systems. The day feed was removed will be referred to as day 0. Days preceding feed removal will be referred to with negative numbers, and days after this event will be referred to with positive numbers.

TABLE 2
Composition of the basal diets

| Ingredient Name | Lbs |
|---|---|
| Corn ground (6.8%) | 824.23 |
| Whey | 500.00 |
| Steam rolled oats | 200.00 |
| HP 300 | 140.00 |
| Bovine plasma | 86.38 |
| Soy protein isolate | 80.00 |
| Choice white grease | 60.00 |
| Fish meal IPC 790 | 60.00 |
| Limestone | 11.32 |
| Kem-gest | 8.00 |
| Salt | 7.00 |
| L-Lysine, 99% | 6.61 |
| DL-Methionine, 99% | 4.23 |
| Monocalcium Phosphate 21% | 3.51 |
| Threonine, 80% | 3.21 |
| Choline chloride, 60% | 1.00 |
| L-Tryptophan, 98% | 1.00 |
| Sow VTM premix | 1.00 |
| Trace mineral premix | 0.67 |
| Selenium, 0.06% | 0.50 |
| Copper sulfate, 25.2% | 0.50 |
| L-Iso | 0.38 |
| Zn sulfate, 35.8% | 0.36 |
| Sel-Plex 3000 | 0.10 |
| Ingredient Total: | 2000.00 |

Nutrient Composition

| | |
|---|---|
| Moisture, % | 9.16 |
| Metabolizable Energy | 1613.00 |
| Swine, kcal/lb | |
| Total sugar, % | 18.20 |
| Crude Protein, % | 20.13 |
| Lysine, % | 1.56 |
| Crude Fat, % | 5.73 |
| Neutral Detergent Fiber, % | 4.68 |
| Lactose, % | 18.60 |
| Ash, % | 5.46 |
| Calcium, % | 0.63 |
| Phosphorus, % | 0.60 |
| Salt, % | 0.35 |

The collection of measurements and biological samples for the trial is outlined in Table 3.

TABLE 3
Measurements and collection of biological samples.

| Event | | Measurement |
|---|---|---|
| Pigs arrived and | | |
| allotted to trts | | |
| −18 | dpfr² | BW¹, fecal, plasma |
| −10 | dpfr | BW, fecal, plasma |
| −3 | dpfr | BW, fecal, plasma |
| Day of Feed Restriction | | BW, fecal, plasma |
| (0 | dpfr) | |
| 1 | dpfr, | BW, fecal, plasma, tissues |
| 23 | pigs harvested | |
| 2 | dpfr | fecal |
| 3 | dpfr | fecal |
| 4 | dpfr, | BW, fecal, plasma, tissues |
| 22 | pigs harvested | |

¹BW = body weight.
²dpfr = days post feed restriction. Negative numbers indicate days prior to feed restriction.

### Results:

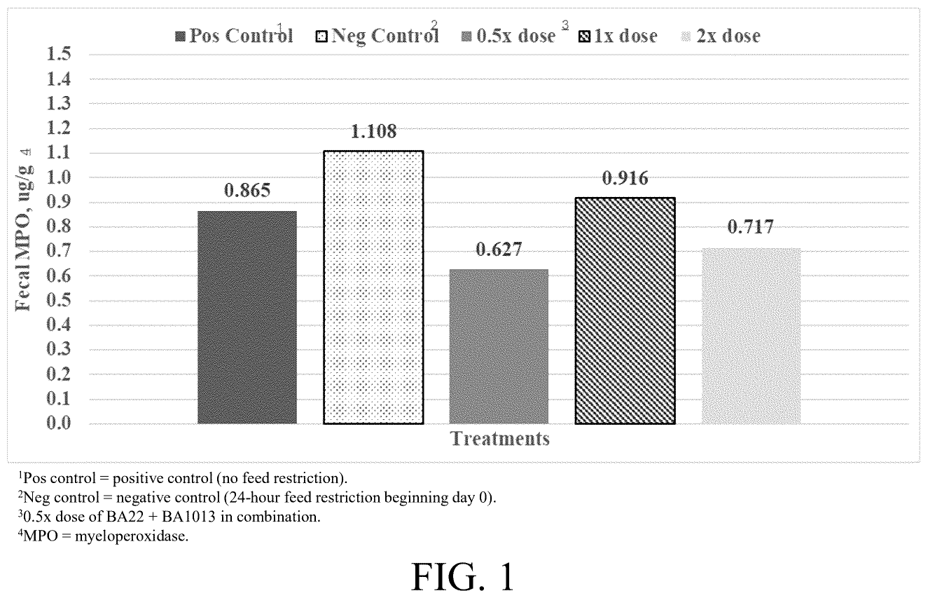

FIG. 1 displays a graph showing the total fecal myeloperoxidase concentrations. Myeloperoxidase (MPO) is an enzyme present in granulocytic and monocytic cells. Increased concentrations of MPO in feces is indicative of intestinal inflammation, which is associated with increased intestinal permeability. The DFM treatment fed at 500,000 CFU/g of feed decreased fecal MPO concentrations compared with both control groups.

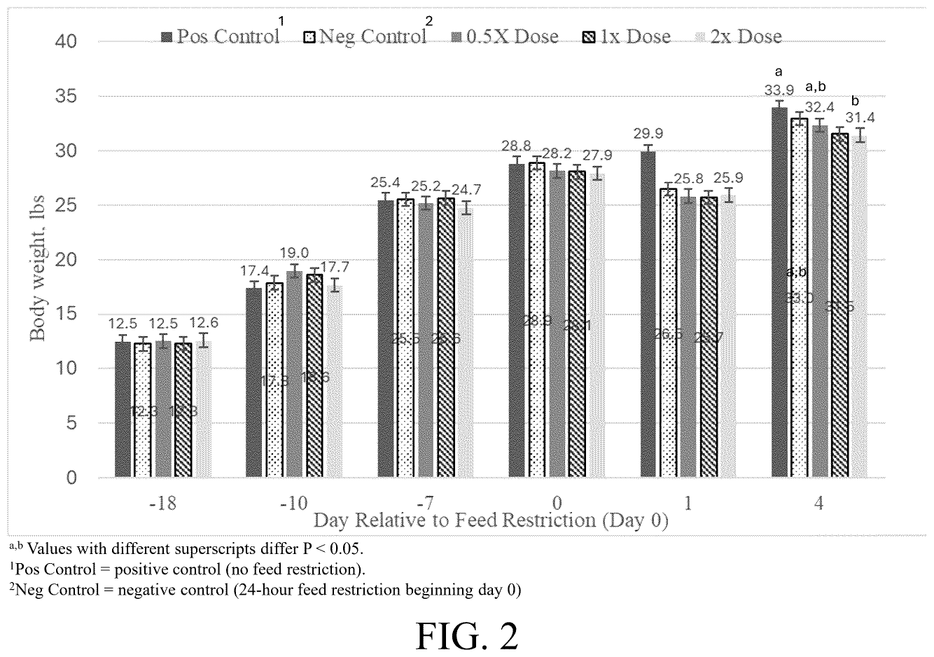

FIG. 2 displays a graph showing the body weight by week of experiment. As shown in FIG. 2, initial body weights were similar for all 5 treatments (FIG. 2). Final body weight was 3.02% higher for the 0.5× dose DFM treatment compared with the other doses of the DFM treatment and was similar to positive control.

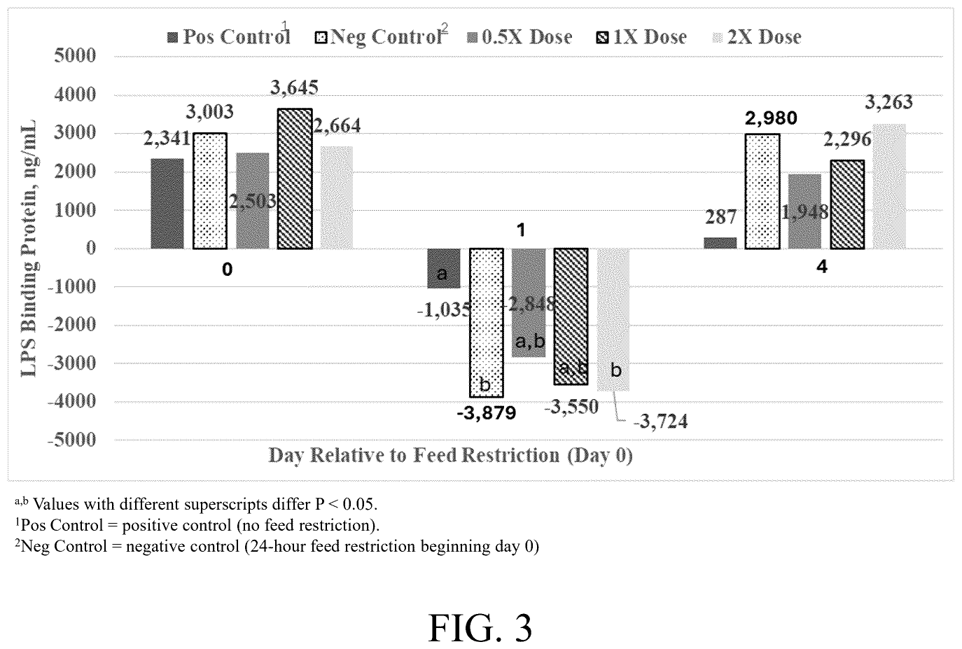

FIG. 3 displays a graph showing the change in lipopolysaccharide binding protein. During periods of stress, LPS concentrations in the blood can increase, which can result in a decrease in LPS binding protein (LBP; Pearce et al., 2014). Twenty-four hours after the feed restriction event, LBP decreased markedly for the groups where feed was removed (treatments 2-5), suggesting an increase in LPS absorption. Analyzing blood samples for LBP is preferred to analyzing for LPS due to the very short half-life of LPS in the blood (Pearce et al., 2014).

TABLE 4
Fold-change in gene expression of ileal tight junction
proteins compared with negative control.

P - value

| | | | 0.5x | 1x | 2x | | | PC vs. |
|---|---|---|---|---|---|---|---|---|
| Item | PC¹ | NC² | dose | dose | dose | SEM | Trt | 4 FR³ |
| Occludin, fold-change NC³ | 1.75 | 1.18 | 1.23 | 1.30 | 1.31 | 0.27 | 0.31 | 0.04 |
| Claudin-3, fold-change NC | 1.75 | 1.24 | 1.34 | 1.05 | 1.35 | 0.32 | 0.33 | 0.06 |
| Tight-junction 1, fold- | 1.63 | 1.09 | 1.39 | 1.31 | 1.43 | 0.40 | 0.76 | 0.33 |
| change NC | | | | | | | | |

¹Pos Control = positive control no feed restriction.
²Neg Control = negative control (24-hour feed restriction beginning day 0).
³FR = feed restriction.

Compared with the positive control, a 24-hour feed restriction event resulted in a decrease in gene expression of ileal tight junction proteins (Table 4), which indicates increased intestinal permeability. This decrease in gene expression was partially alleviated by feeding the DFM regardless of concentration in the diet (Table 4).

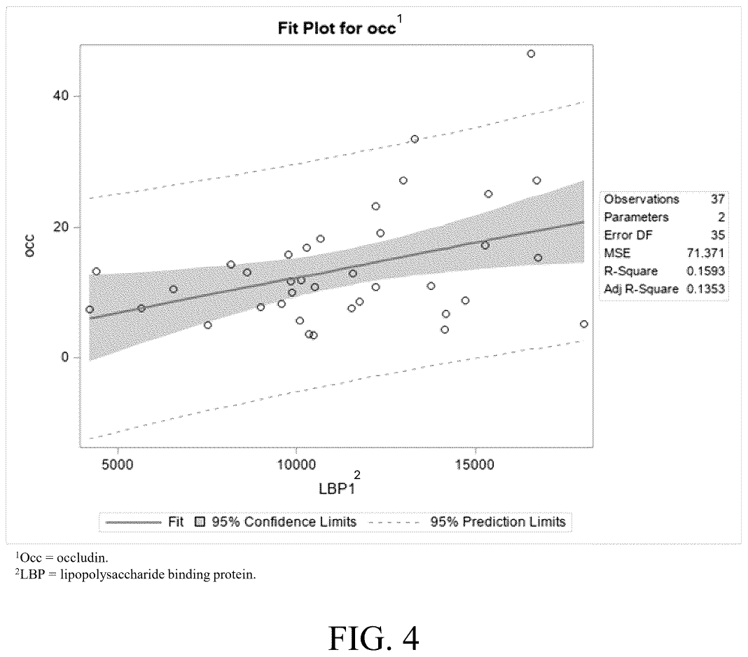

FIG. 4 displays a graph showing the relationship between plasma lipopolysaccharide binding protein concentration and ileal expression of occludin. For animals that were harvested 24 hours post feed restriction (n=37), as LBP concentrations increased, so did gene expression of occludin. This supports the use of plasma LBP concentration as a biomarker for ileal expression of tight junction proteins.

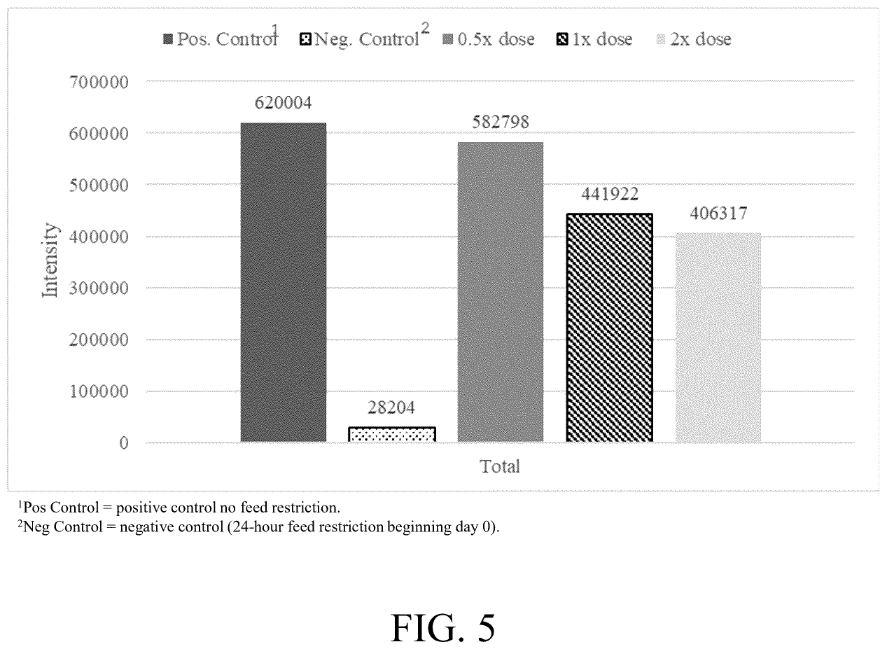

FIG. 5 displays a graph showing the immunohistochemistry staining intensity for Occludin. For a select group of animals (n=25) that were harvested 24 hours post feed restriction, ileal samples were stained for occludin using immunohistochemistry. Intensity of staining was quantified using Image J software. The intensity of staining was much greater for the positive control samples compared with negative control. The 0.5× dose of the DFM had similar intensity for occludin staining to positive control and was over 20 times more intense than the negative control. The other doses of the DFM were intermediate in occludin staining. Higher intensity staining of occludin is indicative of greater expression of tight junction proteins in the intestinal epithelium where tight junction proteins help prevent unwanted absorption of larger molecules such as LPS.

### Example 2. Feeding Green Coffee Bean Extract in Combination with Bacillus amyloliquefaciens Species Fed at 500,000 CFU/g Reduced the Negative Impact of 24-Hour Feed Restriction on Ileal Expression of Tight Junction Proteins and Markers of Inflammation

### Experimental Design & Procedure:

The impact of the disclosed composition on ileal expression of tight junction proteins and inflammation were evaluated. A total of 90 weanling barrows were utilized in this study. Pigs were weighed upon arrival to the facility and housed in 30 pens with 2-3 pigs/pen. Each pen was randomly assigned to one of 5 dietary treatments in a completely randomized design (Table 5).

TABLE 5
Dietary treatments

| | | Diet? Inclusion Rate DFM |
|---|---|---|
| Treatment | Treatment Name | and Green Coffee Bean |
| 1 | Positive Control | NA |
| 2 | Negative Control | NA |
| | (24 hr. feed restriction) | |
| 3 | BA22 + BA1013 0.5x | 5 × 10⁵ CFU/g of feed |
| 4 | BA22 + BA1013 0.5x + | 5 × 10⁵ CFU/g of feed + |
| | Green Coffee Bean Extract | 0.04 lb/ton |
| 5 | Green Coffee Bean Extract | 0.04 lb/ton |

This experiment was conducted for 24 days. Pigs were weaned and arrived at the facility at 26-28 days of age. Upon arrival, pigs were weighed and allotted to one of 30 pens. All pigs were fed the same basal diet balanced to meet requirements (Part 1; Table 6). Treatment 3-5 listed in Table 5 were mixed with the nursery diet that was offered to the pigs ad libitum upon arrival. Treatments 4 and 5 contained green coffee bean extract which fed at the level indicated, supplied 7,560 μg per pig per day. In addition to the feed, pigs were offered ad libitum access to water throughout the trial. After 20 days of receiving treatment diets, feed was removed for a 24-hour period from the pens for pigs assigned to treatments 2-5. This created an off-feed event to simulate stress that occurs naturally in production systems. The day feed was removed will be referred to as day 0. Days preceding feed removal will be referred to with negative numbers, and days after this event will be referred to with positive numbers. Timeline of events, measurements and sample collection is outlined in Table 6.

TABLE 6
Events, measurements, and sample collection.

| Day | Event | Measurement |
|---|---|---|
| −20 | Pigs arrived and | BW¹, plasma, fecal |
| | allotted to treatments | |
| −7 | Sample collection | Plasma, fecal |
| 0 | Feed removed from | BW, plasma, fecal |
| | treatments 2-5 | |
| 1 | 1 pig/pen harvested | BW, plasma, fecal (n = 90), |
| | | Ileal (n = 30) |
| 2 | 1 pig/pen harvested | BW, plasma, fecal (n = 60), |
| | | Ileal (n = 30) |
| 4 | Final 30 pigs harvested | BW, plasma, fecal, |
| | | Ileal (n = 30) |

### Results:

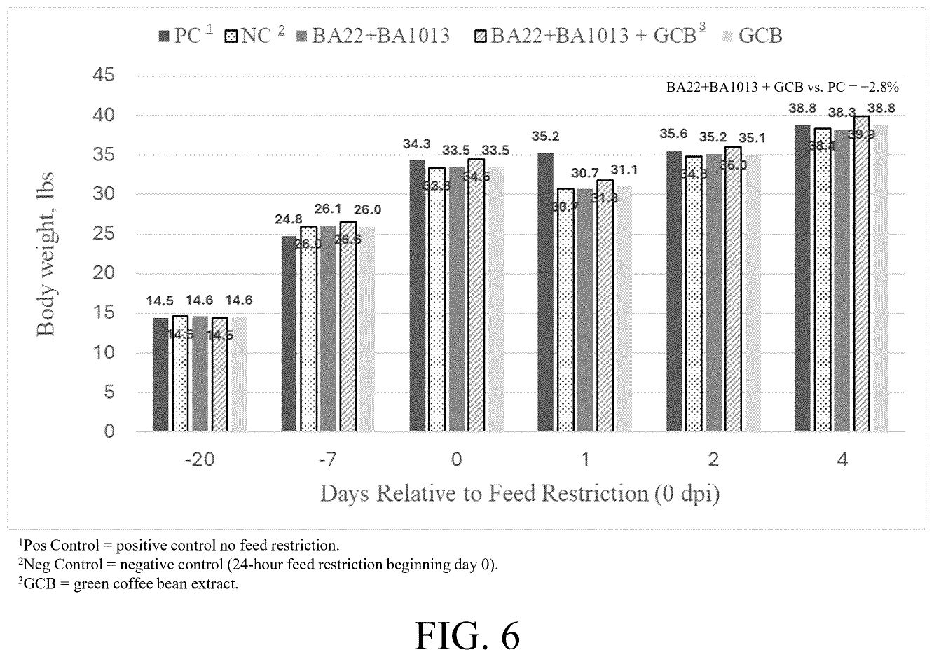

FIG. 6 displays a graph of the body weights by day of experiment. Four days after feed restriction, BW of pigs fed the combination of Bacillus amyloliquefaciens and GCB was greater than positive control by 2.8% (FIG. 6). When fed individually, Bacillus amyloliquefaciens and GCB had no impact on BW 4 days after a feed restriction event.

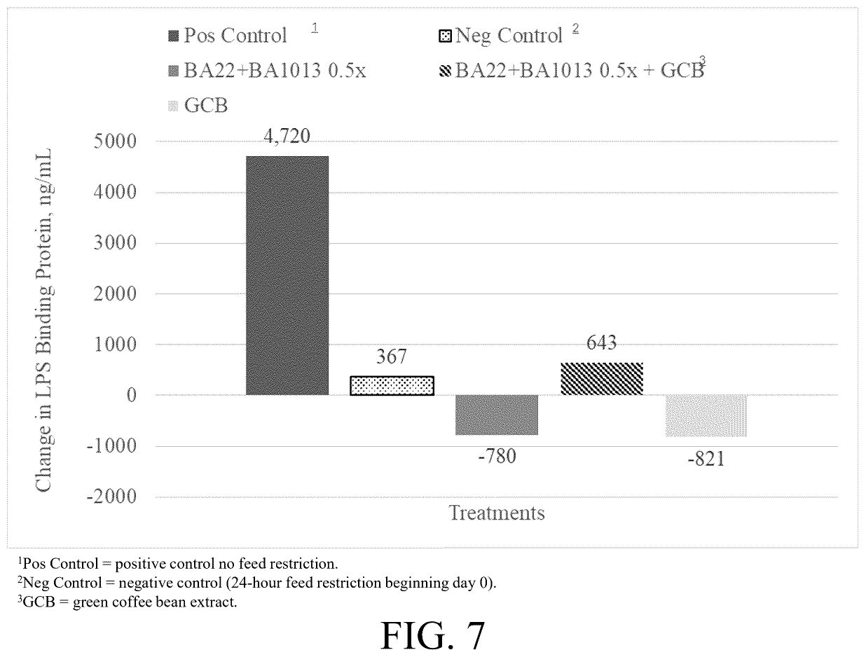

FIG. 7 displays a graph showing LPS Binding Protein 24 hours after feed restriction. After a feed restriction event, LBP decreases in response to increasing LPS in circulation. The DFM combination and GCB prevented the decrease in LBP observed with feed restriction to a greater extent than either product fed alone. The DFM combination and GCB resulted in a positive change in LBP, indicating less intestinal permeability in response to feed restriction, whereas either product fed alone resulted in a negative change in LBP (FIG. 7).

TABLE 7
Fold-change in gene expression of ileal tight junction proteins

P - value

| | | | | | | | | DFM + GCB |
|---|---|---|---|---|---|---|---|---|
| | | | | DFM + | | | | vs. |
| Item | PC | NC | DFM | GCB² | GCB | SEM | Trt | Individual³ |
| Occludin, fold-change | 1.10 | 1.01 | 0.80 | 1.25 | 0.72 | 0.16 | 0.15 | 0.01 |
| NC¹ | | | | | | | | |
| Claudin-3, fold-change | 1.54 | 1.05 | 0.64 | 1.40 | 1.00 | 0.30 | 0.39 | 0.12 |
| NC | | | | | | | | |

¹NC = negative control.
²GCB = green coffee bean extract
³Individual = DFM only or green coffee bean extract alone.

Compared with the individual ingredients, the combination of DFM and GCB improved expression of Occludin and Claudin-3 in ileal tissue harvested from weaned pigs (Table 7), indicating reduced intestinal permeability. This increase in gene expression was similar to the positive control.

### Example 3. Green Coffee Bean Extract in Combination with Bacillus amyloliquefaciens Species Increases Transepithelial Electrical Resistance in IEC-6 Rat Intestinal Cells

### Experimental Design & Procedure:

IEC-6 cells were seeded into 24-well plate hanging inserts coated with type 1 collagen and grown until a monolayer was present. Cells were washed with PBS to remove media containing antibiotics and fresh media was added. Whole Bacillus culture and green coffee bean extract was added. Cells not treated with Bacillus were used as a control. Plates were incubated for 24 hours with measurements taken at 6, 12, and 24 hours. TEER values were measured and calculated according to the manual three times and averaged. Values were normalized to the surface area of the cell monolayer.

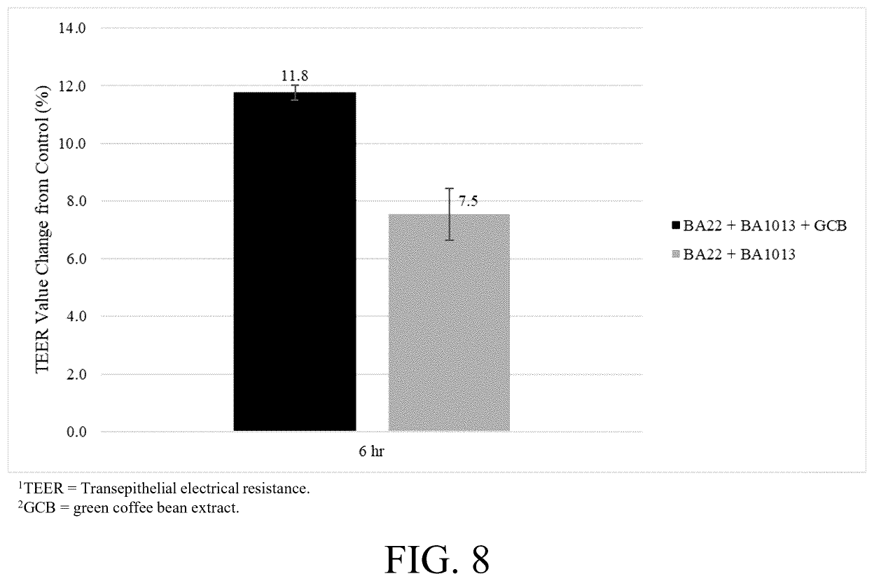

FIG. 8 displays a graph of the transepithelial electrical resistance in IEC-6 rate intestinal cells at 6 hours. Transepithelial electrical resistance (TEER) is a measure of barrier function in intestinal cells. An increased TEER value represents an improved intestinal barrier to prevent unwanted, larger molecules from passing through. Compared with control and BA22+BA1013 alone, adding GCB improved TEER measurements in vitro in a rat intestinal IEC-6 cell line (FIG. 8). With the BA22+BA1013 alone, TEER was about 7% better than control. However, when GCB was added to the combination of Bacillus amyloliquefaciens strains, TEER was nearly 12% better than control.

### Example 4. Green Coffee Bean Extract in Combination with Bacillus velezensis Species Increases Average Daily Gain and Alters Markers of Inflammation Following a Feed Restriction Stress Event

### Experimental Design & Procedure:

The effects of the combination of green coffee bean extract with Bacillus on average daily weight gain and inflammation following a stress event were evaluated. A total of 54 weanling barrows were utilized in this study. Pigs were weighed upon arrival to the facility and housed in 18 pens with 3 pigs/pen. Each pen was randomly assigned to one of 3 dietary treatments in a completely randomized design (Table 8).

This experiment was conducted for 24 days. Pigs were weaned and arrived at the facility at 26-28 days of age. Upon arrival, pigs were weighed and allotted to one of 18 pens. All pigs were fed the same basal diet balanced to meet requirements (Table 9). Treatments 2&3 listed in Table 1 were mixed with the nursery diet (Table 9) that was offered to the pigs ad libitum upon arrival. In addition to the feed, pigs were offered ad libitum access to water throughout the trial. After 20 days of receiving treatment diets, feed was removed for a 24-hour period from the pens for pigs assigned to treatments 2 and 3. This created an off-feed event to simulate stress that occurs naturally in production systems. The day feed was removed will be referred to as day 0. Days preceding feed removal will be referred to with negative numbers, and days after this event will be referred to with positive numbers.

TABLE 8
Dietary Treatments

| Treatment | Treatment Name | Inclusion Rate |
|---|---|---|
| 1 | Positive Control | NA |
| 2 | ZBG17 & C&D1781¹ | ZBG17 + C&D1781; |
| | | 250,000 CFU/g of |
| | | feed each |
| 3 | ZBG17 & C&D178 + | ZBG17 + C&D1781; |
| | GCB² | 250,000 CFU/g of |
| | | feed each + |
| | | GCB 14.3 mg/pig/d |

¹ZBG17 = Bacillus velezensis and CD1781 = Bacillus subtilis.
²GCB = green coffee bean extract.

TABLE 9
Composition of the basal diets

| | Leaky |
|---|---|
| | Gut |
| Ingredient Name | Diet |
| Corn ground (6.8%) | 824.23 |
| Whey | 500.00 |
| Steam rolled oats | 200.00 |
| HP 300 | 140.00 |
| Bovine plasma | 86.38 |
| Soy protein isolate | 80.00 |
| Choice white grease | 60.00 |
| Fish meal IPC 790 | 60.00 |
| Limestone | 11.32 |
| Kem-gest | 8.00 |
| Salt | 7.00 |
| L-Lysine, 99% | 6.61 |
| DL-Met, 99% | 4.23 |
| Monocalcium Phosphate 21% | 3.51 |
| Threonine, 80% | 3.21 |
| Choline chloride, 60% | 1.00 |
| L-Tryptophan, 98% | 1.00 |
| Sow VTM premix | 1.00 |
| Trace mineral premix | 0.67 |
| Selenium, 0.06% | 0.50 |
| Copper sulfate, 25.2% | 0.50 |
| L-Isolatel | 0.38 |
| Zinc sulfate, 35.8% | 0.36 |
| Sel-Plex 3000 | 0.10 |
| Ingredient Total: | 2000.00 |

Nutrient Composition

| | |
|---|---|
| Moisture, % | 9.16 |
| Metabolizable Energy Swine, kcal/lb | 1613.00 |
| Total sugar, % | 18.20 |
| Crude Protein, % | 20.13 |
| Lysine, % | 1.56 |
| Crude Fat, % | 5.73 |
| Neutral detergent fiber, % | 4.68 |
| Lactose, % | 18.60 |
| Ash, % | 5.46 |
| Calcium, % | 0.63 |
| Phosphorus, % | 0.60 |
| Salt, % | 0.35 |

The collection of measurements and biological samples for the trial is outlined in Table 10.

TABLE 10
Measurements and collection of biological samples.

| Date | DPFR¹ | Event | Measurement |
|---|---|---|---|
| Jul. 3, 2024 | −20 | Pigs arrived and | BW², plasma, fecal |
| | | allotted to | |
| | | treatments | |
| Jul. 10, 2024 | −7 | Sample collection | Plasma, fecal |
| Jul. 22, 2024 | 0 | Feed removed from | BW, plasma, fecal |
| | | treatments 2-5 | |
| Jul. 23, 2024 | 1 | 2 pig/pen harvested | BW, plasma, fecal, |
| | | | Ileal, liver, |
| Jul. 26, 2024 | 4 | Final pigs harvested | BW, plasma, fecal, |
| | | | Ileal, liver |

¹DPFR = days post feed restriction. Negative number is before feed restriction, 0 is day of feed restriction.
²BW = body weight.

### Results:

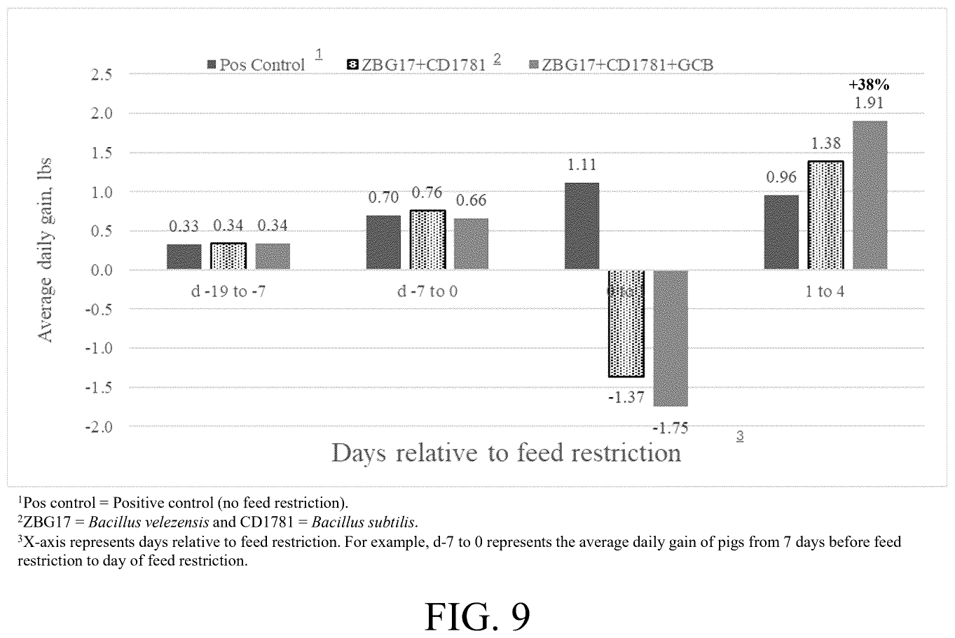

FIG. 9 displays a graph of the average daily gain four days following a feed restriction event. Following a feed restriction event, piglets lose weight due to changes in gut fill. Once feed is reintroduced to the pigs, average daily gain increases as gut fill is resumed and compensatory gain is observed. Adding a combination of 2 Bacillus DFM species showed increased ADG compared with the positive control (no feed restriction) and adding GCB to these Bacillus species improved ADG even further (increased by 38%) than the Bacillus alone following a feed restriction event.

TABLE 11
Change in lipopolysaccharide binding protein
following a 24-hour feed restriction event.

| | | | ZBG17 + | | |
|---|---|---|---|---|---|
| | Positive | ZBG17 + | CD1781 + | | |
| Item¹ | Control | CD1781 | GCB² | SE | P ≤ |
| LBP change, ng/ml | 1084.8ª | −4608.7^{b} | −1022.3^{a,b} | 882.4 | 0.0004 |

^{a,b}Values with different superscripts differ P < 0.10
¹LPS = lipopolysaccharide.
²ZBG17 = Bacillus velezensis strain and CD1781 = Bacillus subtilis; GCB = green coffee bean extract.

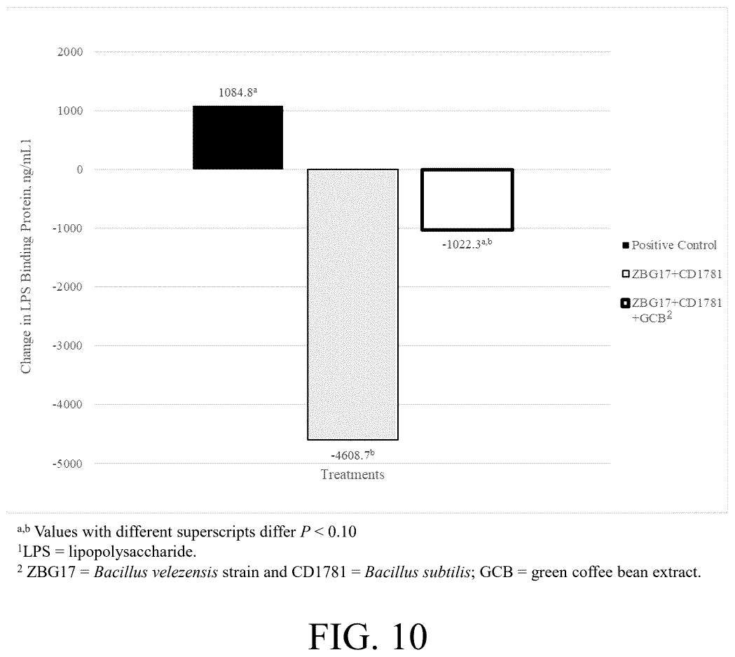

FIG. 10 displays a graph showing the change in lipopolysaccharide binding protein following a 24-hour feed restriction event. Following a feed restriction even, LPS binding protein decreases in plasma. Feeding these Bacillus strains prior to the feed restriction event did not improve LPS binding protein in piglets compared with positive control. However, when GCB extract was added in addition to the DFM strains, LPS binding protein was similar to positive control following a feed restriction event (Table 11).

### Example 5. Green Coffee Bean Extract in Combination with Bacillus amyloliquefaciens and Bacillus velezensis Species Increases Feed Efficiency and Alters Markers of Inflammation Following an Enterotoxigenic E. Coli (ETEC) Challenge

The effects of green coffee bean extract in combination with Bacillus subtilis in response to an enterotoxigenic E. Coli challenge were evaluated. This experiment was conducted for 22 days. Pigs were weaned and arrived at the facility at 26-28 days of age. Upon arrival, pigs were weighed and allotted to one of 18 pens. All pigs were fed the same basal diet balanced to meet requirements. Treatments of 1) GCB, 2) GCB+BA22+BA1013, and 3) GCB+ZBG17+CGS1.1 were mixed with the nursery diet that was offered to the pigs ad libitum upon arrival. In addition to the feed, pigs were offered ad libitum access to water throughout the trial. After 8 days of receiving treatment diets, on day 0, each pig was orally inoculated with 5 ml (approximately 2.0×10⁹/mL with a total challenge of 10¹⁰ CFU) of freshly grown E. coli F18 inoculants (derived from approximately year 2015 strain) via a polyethylene tube attached to a syringe placed in the back of the oral cavity. Measurements of body weight, feed intake, and diarrhea score were made post-challenge to determine if the dietary treatments had an impact on animal performance and health following and ETEC challenge. Serum samples were also collected to determine LPS binding protein concentrations following an ETEC challenge. An uninoculated control group served as the positive control.

TABLE 11
Response to enterotoxigenic E. coli challenge when fed a combination
of green coffee bean extract and Bacillus species.

| ETEC Challenge: | | | | | | |
|---|---|---|---|---|---|---|
| No | Yes | Yes | Yes | Yes | | |
| Treatment: | | | | | | |
| | | Green | GCB1 + | | | |
| | | coffee | BA22 + | GCB + ZBG17 + | | P- |
| Control | Control | bean | BA1013 | CGS1.1 | SE | Value |

LPS2 Binding Protein Concentration, ng/ml (with D 0 as co-variate)

| | | | | | | | |
|---|---|---|---|---|---|---|---|
| D 4 | 9010 | 9948 | 11571 | 8991 | 10075 | 1267.4 | 0.580 |
| Change d 4-d 0 | −3326 | −2387 | −764 | −3344 | −2261 | 1267.4 | 0.580 |
| Gain:Feed | 0.44b | −0.82^{a} | −1.24^{a} | −0.70^{a} | −0.65^{a} | 0.390 | 0.020 |
| Diarrhea score, | 1.76^{ab} | 1.33^{a} | 2.43^{b} | 2.08^{b} | 1.74^{ab} | 0.249 | 0.005 |
| Day 2 | | | | | | | |

^{a, b}Values with different superscripts differ P < 0.05.
¹GCB = Green coffee bean extract.
²LPS = lipopolysaccharide.

Following an ETEC challenge, pigs fed GCB in combination with different Bacillus strains had lower LPS binding protein concentrations compared with feeding GCB alone (Table 11). Also, the change in LPS binding protein was similar to positive control for the GCB+Bacillus combinations. Unlike an acute feed restriction challenged, during a pathogenic infection, LPS binding protein is a marker of inflammation, and increased LPS binding protein can signal greater inflammatory status. In this instance, inflammation was decreased in the face of the challenge when GCB was fed in combination with Bacillus. Additionally, diarrhea scores improved 2 days after the challenge with the GCB and Bacillus treatments and feed efficiency was improved through 14 days after the challenge. The combination of GCB and Bacillus strains not only improves gut integrity during a feed restriction stress event, but also decreases inflammation and improves performance after an ETEC challenge.

### Example 6. Green Coffee Bean Extract in Combination with Bacillus amyloliquefaciens Species Increases Milk Yield without a Negative Impact on Body Condition or Body Weight Change Through the First 90 Days of Lactation

### Experimental Design and Facilities:

The effects of the administration of green coffee bean extract in combination with Bacillus amyloliquefaciens on cow lactation were evaluated. Ninety pregnant Holstein cows entering second or greater lactation were enrolled in a randomized complete block design and assigned to one of two dietary treatments: 1) Control—no product and 2) Leaky Gut Product B(BA22+BA1013 2×10⁹ CFU/d+Green Coffee Bean extract 400 mg/d). Animals received treatments beginning at 21 days before expected calving date through 90 days in milk. Animals were blocked by orientation of robot in previous lactation (left or right), calving date, parity (2nd or 3rd+), and previous lactation 305 ME milk yield.

TABLE 12
Ingredient nutrient composition of Close-Up and Lactating Pellets.

| | Close-up | Close-up | Lactating | Lactating |
|---|---|---|---|---|
| | Control | Leaky | Control | Leaky |
| | % of | Gut | % of | Gut |
| Ingredient | DM | % of DM | DM | % of DM |
| Corn grain, ground fine | 33.98 | 33.65 | 33.76 | 33.72 |
| Wheat middlings | 28.34 | 27.59 | 28.77 | 28.80 |
| Soybean meal, | 10.15 | 11.16 | 13.89 | 13.93 |
| solvent 47.5 | | | | |
| Amino Plus | 9.92 | 9.92 | 10.67 | 10.65 |
| Soybean hulls, ground | 14.87 | 14.87 | 8.71 | 8.7 |
| Molasses, beet | 1.9 | 1.9 | 1.27 | 1.27 |
| International Ingredients | — | — | 1.57 | 1.61 |
| Sugar | | | | |
| Tallow, porcine | 0.84 | 0.84 | 1.08 | 1.11 |
| Maxi Bond | — | — | 0.10 | 0.11 |
| Super Lube | — | — | 0.10 | 0.05 |
| BA22 + BA1013 + GCB¹ | — | 0.08 | — | 0.06 |

Nutrient Composition of Pellets Item

| | | | | |
|---|---|---|---|---|
| Dry matter (DM), % | 88.09 | 88.17 | 87.74 | 87.79 |
| Crude protein, % DM | 21.25 | 22.42 | 22.79 | 23.05 |
| Neutral detergent | 23.73 | 24.22 | 20.58 | 20.14 |
| fiber, % DM | | | | |
| Starch, % DM | 28.45 | 25.80 | 29.49 | 28.76 |
| Sugar, % DM | 8.47 | 10.06 | 11.65 | 10.63 |
| Ether extract, % DM | 4.76 | 4.40 | 4.79 | 4.57 |

¹BA22 + BA1013 = 2 strains of Bacillus amyloliquefaciens; GCB = green coffee bean extract.

TABLE 13
Milk yield, pellet intake, milking frequency, milking duration, and body weight.

| | | LG² | | | | Trt* |
|---|---|---|---|---|---|---|
| Item¹ | Control | Product | SE | Trt | Wk | Wk³ |
| Milk Yield, lbs/d | 113.49 | 117.61 | 1.44 | <0.05 | <0.0001 | 0.28 |
| Pre-fresh Pellet Intake, lbs/d | 3.55 | 3.51 | 0.03 | 0.36 | <0.0001 | 0.64 |
| Pellet Intake, lbs/d | 11.68 | 11.27 | 0.63 | 0.65 | <0.0001 | 0.72 |
| Number of Milkings/d | 2.53 | 2.65 | 0.07 | 0.21 | <0.0001 | 0.003 |
| Pre-calving BCS | 3.63 | 3.67 | 0.04 | 0.50 | <0.0001 | 0.34 |
| Post-calving BCS | 3.06 | 3.11 | 0.04 | 0.37 | <0.0001 | 0.20 |
| Initial BW (−21 d), lbs | 1813.46 | 1807.56 | 13.10 | — | — | — |
| BW, lbs | 1652.58 | 1642.65 | 10.21 | 0.50 | <0.0001 | 0.04 |
| BW change 1-90 DIM | −104.32 | −130.78 | 15.57 | 0.26 | N/A | N/A |

^{a}Values with different superscripts differ, P < 0.05.
^{x}Values with different superscripts tend to differ, P <0.10
¹BCS-body condition score; BW = body weight; DIM = days in milk.
²LG = leaky gut; trt = treatment; wk = weeks.

Milk yield was improved on average by 4 lbs per cow per day in the first 90 days of lactation with supplementing the leaky gut product in the feed. (Table 13). There was no difference among treatments in body condition score (BCS) or body weight (BW) change throughout the trial, which indicates that the leaky gut product was enhancing dietary nutrient utilization. The cows consuming the leaky gut product also did not eat more pellet, which may indicate an improvement in feed efficiency.

It is desirable to not only increase milk yield, but also increase the yield of fat and protein. Energy-corrected milk yield (ECM) is used to standardize milk for components and is calculated using the following equation: (0.327*Milk Yield)+(12.95*Fat Yield)+(7.2*Protein Yield). Supplementing the leaky gut product in the feed of lactating dairy cows before expected calving date through 90 days in lactation resulted in 0.3 lbs of more milk protein produced per cow per day and 4.64 lbs of ECM yield produced per cow per day (Table 14).

TABLE 14
Daily milk component and energy-corrected milk yields.

P -value

| | | LG² | | | | |
|---|---|---|---|---|---|---|
| Item¹ | Control | Product | SE | Trt | Week | Trt*Wk³ |
| Fat Yield, lbs | 4.76 | 4.88 | 0.09 | 0.38 | <0.0001 | 0.24 |
| Protein Yield, lbs | 3.20 | 3.50 | 0.07 | 0.04 | <0.0001 | 0.89 |
| ECM Yield, lbs | 125.70 | 130.34 | 1.80 | 0.07 | <0.0001 | 0.39 |

¹ECM = energy-corrected milk.
²LG = leaky gut.
³Trt = treatment; wk = week.

In addition to production responses outlined above, feeding the leaky gut product to lactating dairy cows resulted in fewer health events post-calving. The transition period in modern dairy production is defined as the three weeks prior to expected calving date to 3 weeks post-calving. There are many metabolic diseases that can occur during this period due to the increased nutrient demands of start-up lactation. These metabolic disorders include hypocalcemia, ketosis, and retained placenta, among others. Looking at the percentage of cows with a documented health events (including metabolic disorders mentioned above) in the current study, cows that were fed leaky gut product had fewer health events than the control animals (Table 4). Over 23% of the control cows presented with a recorded health event post-calving. Fewer than 10% of the cows fed the test article had a recorded health event (Table 15).

TABLE 15
Health Events, Culling, and Reproduction
through 13 weeks of lactation

| | | | P≤ |
|---|---|---|---|
| Item | Control | LG Product | Treatment |
| Health Event, % | 23.81 | 9.09 | 0.06 |
| Cull, % | 7.14 | 4.55 | 0.49 |
| Pregnant, % | 37.93 | 44.44 | NS |

| | |
|---|---|
| Hazard ratios | Cont. vs Leaky |
| (95% CI) | gut Product |
| Health Event | 3.10 (0.96-10.00) |
| Cull | 1.88 (0.31-11.27) |

Lipopolysaccharide (LPS) binding protein (LBP) is a marker of intestinal integrity and inflammation during lactation. The early lactation period of a cow is marked by increases in inflammation to prepare for lactation and to resolve uterine involution post-calving. Feeding the leaky gut product decreased plasma LBP concentrations by 4.32% (Table 16). The absolute change in LBP can also indicate a lower inflammatory status. Feeding the leaky gut product resulted in 4.67% lower change in LBP after calving, indicating an improved inflammatory status, and suggesting improved gut integrity taken with the data in the nursery pigs presented previously.

Fecal IgA is a biomarker of gut health playing a critical role in the mucosal barrier. Higher fecal IgA concentrations indicate improved intestinal integrity by helping to protect the intestinal barrier and prevent unwanted bacteria or toxins from entering the body. Feeding the leaky gut product increased fecal IgA by 146%.

In early lactation, lower plasma glucose concentrations in multiparous cows are reported to have a positive impact on milk production through 100 days in milk (Ruoff et al., 2017). Feeding the leaky gut product resulted in 5% lower plasma glucose concentrations through 21 days in milk. Taken together with the LBP and fecal IgA data, these indicate that the leaky gut product is improving overall intestinal integrity and leading to a more favorable metabolic state that allows for improved health and performance, as shown through improvements in health events, milk yield and components through 90 DIM.

TABLE 16
Leaky gut product improves biomarkers of intestinal integrity and milk production.

P -value

| | | LG² | | | | |
|---|---|---|---|---|---|---|
| Item | Control | Product | SE | Trt³ | day | Trt*day⁴ |
| LPS Binding Protein, ng/mL | 8106 | 7882 | 388.64 | 0.67 | <0.0001 | 0.53 |
| LBP1 change from −21 DIM | 3811 | 3607 | 359.70 | 0.73 | <0.0001 | 0.78 |
| LBP change −10 to 1 DIM | 5266 | 4131 | 227.30 | 0.001 | NA | NA |
| LBP change from −10 to 5 | 10733 | 9507 | 374.90 | 0.03 | NA | NA |
| DIM | | | | | | |
| Fecal IgA, ng/mL | 4.16 | 60.56 | 143.46 | 0.007 | NA | NA |
| Glucose, mg/dL | 75.48 | 71.17 | 0.75 | <0.0001 | <0.001 | 0.77 |

¹LBP = lipopolysaccharide binding protein.
²LG = leaky gut.
³Trt = treatment.
⁴Day of sampling included in analysis −10, 1, 5, and 10 days in milk. Change is represented from these davs versus −21 DIM.

Smaller changes in plasma LBP from 10 days before calving to 5 days in milk, lower plasma glucose concentrations at 10 days in milk, and lower plasma glucose concentrations at 21 days in milk were associated with increased milk and energy-corrected milk yields using a multiple linear regression model with a step-wise forward selection procedure (Table 17). All of these parameters were lower with feeding the leaky gut product and milk production was also improved with feeding the leaky gut product

TABLE 17
Relationship between biomarkers and milk and energy-corrected milk yield.

| Item | Σ R² | y-intercept | Slope | SE | Partial R² | P≤ |
|---|---|---|---|---|---|---|
| Milk Yield, lbs | 0.20 | 178.50 | | | | |
| LBP change from −10 to 5 DIM | | | −0.000 | 0.00 | 0.11 | 0.004 |
| Glucose 10 DIM, mg/dL | | | −0.47 | 0.20 | 0.07 | 0.02 |
| Glucose 21 DIM, mg/dL | | | −0.17 | 0.18 | 0.03 | 0.11 |

195.07

| | | | | | | |
|---|---|---|---|---|---|---|
| Energy-corrected milk yield, lbs | 0.22 | 195.07 | | | | |
| Glucose 10 DIM, mg/dL | | | −0.52 | 0.19 | 0.12 | 0.002 |
| LBP change from −10 to 5 DIM | | | −0.00 | 0.00 | 0.07 | 0.02 |
| Glucose 21 DIM, mg/dL | | | −0.30 | 0.17 | 0.03 | 0.08 |

¹LBP = lipopolysaccharide binding protein.
²LG = leaky gut.
³Trt = treatment.

### Example 6. Control of Intestinal Integrity in Poultry Using Green Coffee Bean Extract in Combination with Bacillus amyloliquefaciens Species

In a controlled broiler study, the effects of combining Green Coffee Bean (GCB) extract (containing 50% chlorogenic acid) with Bacillus amyloliquefaciens species (DFM) were evaluated against necrotic enteritis (NE) induced by a Clostridium perfringens (C.P.) challenge. The study was designed to simulate commercial poultry production conditions, including vaccination with a coccidiosis vaccine and subsequent C. perfringens exposure.

A total of 1,900 male Ross broiler chicks were randomized into 6 treatment groups and placed in floor pens with new litter (Table 18). Among the treatments, the DFM+GCB group at 1.5×10⁵ CFU/g plus 25 ppm GCB was compared to both a challenge control group (no treatment) and a group receiving bacitracin methylene disalicylate (BMD), a commonly used antibiotic in broilers.

TABLE 18
Study Design

| | | | | Starter | Grower | Finisher |
|---|---|---|---|---|---|---|
| | | | C.P. | (kg/Metric | (kg/Metric | (kg/Metric |
| Group | Treatment* | Reps | Challenge** | ton) | ton) | ton) |
| 1 | Challenge Control | 12 | Yes | — | — | — |
| 2 | DFM (5.0 × | 13 | Yes | DFM = 0.33 | DFM = 0.33 | DFM = 0.33 |
| | 10⁵ cfu/g) + | | | GCB = 0.025 | GCB = 0.025 | GCB = 0.025 |
| | Green Coffee | | | | | |
| | Bean¹ | | | | | |
| 3 | DFM (1.5 x | 13 | Yes | DFM = 0.10 | DFM = 0.10 | DFM = 0.10 |
| | 10⁵ cfu/g) + | | | GCB = 0.025 | GCB = 0.025 | GCB = 0.025 |
| | Green Coffee | | | | | |
| | Bean¹ | | | | | |
| 4 | DFM (7.5 x | 13 | Yes | DFM = 0.05 | DFM = 0.05 | DFM = 0.05 |
| | 10⁴cfu/g) + | | | GCB = 0.0125 | GCB = 0.125 | GCB = 0.125 |
| | Green Coffee | | | | | |
| | Bean¹ | | | | | |
| 5 | BMD | 12 | Yes | 55 gms/mTon | 55 gms/mTon | 55 gms/mTon |

*All birds were vaccinated with Merck Coccivac B52 on Day 0 at 1 dose/1 bird.
**C.P. challenged days 14 and 15
¹Green coffee bean extract contained 50% chlorogenic acid

### Result 1—Evidence of Improved Gut Integrity—FITC-d Assay (Table 19)

Serum FITC-dextran (FITC-d) levels were assessed at Day 16 post-challenge to evaluate gut permeability. As FITC-d is a non-digestible marker, elevated levels in blood indicate compromised intestinal barrier function.

Compared to the Control group, combinations of Green Coffee Bean extract with various levels of DFM showed 1.2-6.4% reduction in serum FITC-d. The high-dose DFM (5×10⁵ CFU/g)+GCB group showed a similar result on FITC-d as the BMD group.

The reductions in plasma FITC-d support the ability of the DFM+GCB combination to reduce gut leakage and improve gut integrity associated with NE.

TABLE 19
Serum FITC-d Results on Day 16 by Treatment

| | Treatment | | | | |
|---|---|---|---|---|---|
| | | DFM | DFM | DFM | |
| | | (5.0 × 10\hat{ }5 | (1.5 × 10\hat{ }5 | (7.5 × 10\hat{ }4 | |
| | Challenge | cfu/g) + Green | cfu/g) + Green | cfu/g) + Green | |
| Item | Control | Coffee Bean | Coffee Bean | Coffee Bean | BMD |
| FITC-d, μg/ml | 1.712 | 1.603 (−6.4%) | 1.691 (−1.2%) | 1.613 (−5.8%) | 1.601 (−6.5%) |

### Result 2—Reduced Clinical NE—Mortality and Lesion Scores (Table 20)

The challenge control group had 13.3% NE mortality, whereas the DFM+GCB (1.5×10⁵ CFU/g) group had 7.7%, closely matching the BMD group at 5.7%.
Lesion scores in the DFM+GCB groups were numerically lower than the challenge control, indicating milder intestinal damage.

TABLE 20
Clinical NE Results

| | | Lesion Scores | | |
|---|---|---|---|---|
| | | | (Non parametric | |
| | | | analysis) | |
| | | | Kruskal- | |
| | | | Wallis | Dunn's |
| | | Mean | One-Way | All- |
| | NE | Score | AOV | Pairwise |
| | Percent | (Hofacre | (Mean | Comparisons |
| Treatment | Mortality | 1998) | Rank) | Test |
| 1. Challenge Control | 13.333 | 0.583 | | |
| 2. DFM (5.0 × 10\hat{ }5 cfu/g) + | 10.154A | 0.539A | 26.50 | 26.50A |
| Green Coffee Bean | | | | |
| 3. DFM (1.5 × 10\hat{ }5 cfu/g) + | 7.692A | 0.462A | 24.58 | 24.58A |
| Green Coffee Bean | | | | |
| 4. DFM (7.5 × 10\hat{ }4 cfu/g) + | 11.385A | 0.692A | 31.31 | 31.31A |
| Green Coffee Bean | | | | |
| 5. BMD | 5.667 | 0.667 | | |

### Result 3—Improved Growth Performance Compared to Control (Table 21)

The DFM+GCB 1.5×10⁵ CFU/g) group achieved a body weight gain of 3.043 kg, nearly identical to the BMD group at 3.044 kg.
The same DFM+GCB group also showed a significantly improved feed conversion ratio (FCR) compared to Challenge Control
Mortality in the DFM+GCB group was reduced to 11.1%, compared to 16.3% in the challenge control.

These results demonstrate that the DFM+GCB treatment not only maintained performance under NE challenge but also approached or matched the effectiveness of the antibiotic (BMD) group.

TABLE 21
Day 0 to 43 Performance Results

| | Feed Intake | Adjusted | Non-Adjusted | Weight | Percent |
|---|---|---|---|---|---|
| Treatment | (Kg/Pen) | FCR* | FCR | Gain (kg) | Mortality |
| 1. Challenge Control | 92.979 | 1.471 | 1.524 | 3.040 | 16.333 |
| 2. DFM (5.0 × 10\hat{ }5 cfu/g) + | 93.677A | 1.462AB | 1.507AB | 3.036A | 14.154A |
| Green Coffee Bean | | | | | |
| 3. DFM (1.5 × 10\hat{ }5 cfu/g) + | 96.512A | 1.458B | 1.492B | 3.043A | 11.077A |
| Green Coffee Bean | | | | | |
| 4. DFM (7.5 × 10\hat{ }4 cfu/g) + | 91.312A | 1.470AB | 1.519AB | 2.977A | 15.385A |
| Gree nCoffee Bean | | | | | |
| 5. BMD | 97.758 | 1.450 | 1.479 | 3.044 | 10.000 |

*Adjusted FCR is adjusted for mortality

In conclusion, this study provides strong evidence that a combination of Green Coffee Bean extract with Bacillus amyloliquefaciens species DFM enhances gut barrier function, reduces NE-associated mortality and lesion severity, maintains growth performance and feed efficiency, and serves as a viable non-antibiotic alternative to conventional antibiotics such as BMD. This combination may be used to support gut health and performance in poultry, particularly under intestinal challenge conditions such as necrotic enteritis.

### Example 7

Ninety pregnant Holstein cows entering second or greater lactation were enrolled in a randomized complete block design and assigned to one of two dietary treatments: 1) Control—no product and 2) Leaky Gut Product (BA22+BA1013 2×109 CFU/d+Green Coffee Bean extract 400 mg/d). Animals received treatments beginning at 21 days before expected calving date through 90 days in milk. Animals were blocked by orientation of robot in previous lactation (left or right), calving date, parity (2nd or 3rd+), and previous lactation 305 ME milk yield.

TABLE 22
Ingredient and nutrient composition of Close-Up and Lactating Pellets.

| | Close-up | Close-up | Lactating | |
|---|---|---|---|---|
| | Control | LG | Control | Lactating |
| | Pellet | Pellet | Pellet | LGPellet |
| Ingredient | % of DM | % of DM | % of DM | % of DM |
| Corn grain, ground fine | 33.98 | 34.18 | 33.65 | 33.71 |
| Wheat midds | 27.84 | 27.84 | 28.80 | 28.80 |
| Soybean meal, solvent 47.5 | 10.14 | 10.14 | 13.93 | 13.93 |
| Amino Plus | 9.92 | 9.92 | 10.65 | 10.65 |
| Soybean hulls, ground | 14.98 | 14.98 | 8.70 | 8.70 |
| Molasses Beet | 1.90 | 1.90 | 1.27 | 1.27 |
| Tallow, porcine | 0.84 | 0.84 | 1.11 | 1.11 |
| Maxi Bond | — | — | 0.11 | 0.11 |
| Super Lube | — | — | 0.05 | 0.05 |
| Sotera | — | 0.20 | — | 0.06 |

Nutrient Composition of Pellets Item

| | | | | | | | | |
|---|---|---|---|---|---|---|---|---|
| DM, % | 87.66 | (±0.36) | 87.89 | (±0.98) | 87.18 | (±0.32) | 87.88 | (±0.53) |
| CP, % DM | 22.47 | (±1.18) | 21.12 | (±0.49) | 22.22 | (±0.65) | 22.95 | (±0.27) |
| aNDFom, % DM | 23.33 | (±0.71) | 24.60 | (±0.34) | 21.11 | (±0.59) | 20.85 | (±0.65) |
| Starch, % DM | 29.10 | (±0.76) | 28.61 | (±0.88) | 28.74 | (±0.90) | 28.01 | (±0.96) |
| Sugar, % DM | 9.99 | (±0.78) | 9.66 | (±1.00) | 12.10 | (±0.63) | 12.21 | (±0.97) |
| Ether extract, % DM | 4.59 | (±0.66) | 4.63 | (±0.42) | 5.58 | (±0.54) | 5.13 | (±0.46) |

TABLE 23
Milk yield, pellet intake, milking frequency, milking duration, and body weight.

| | | LG | | | | Trt* |
|---|---|---|---|---|---|---|
| Item | Control | Product | SE | Trt | Wk | Wk |
| Milk Yield, lbs/d | 111.43 | 117.21 | 1.76 | 0.02 | <0.0001 | 0.44 |
| Pre-fresh Pellet Intake, lbs/d | 3.50 | 3.63 | 0.05 | 0.08 | <0.0001 | 0.24 |
| Pellet Intake, lbs/d | 11.93 | 11.80 | 0.40 | 0.81 | <0.0001 | 0.03 |
| Number of Milkings/d | 2.60 | 2.48 | 0.08 | 0.32 | 0.001 | 0.26 |
| Milking duration, sec/milking | 462.22 | 498.49 | 13.99 | 0.07 | 0.001 | 0.66 |
| Post-calving BCS | 2.87 | 2.90 | 0.04 | 0.65 | <0.0001 | 0.40 |
| Initial Body Weight (−21 d), lbs | 1753.13 | 1774.64 | — | — | — | — |
| BW, lbs | 1607.47 | 1599.62 | 12.70 | 0.56 | <0.0001 | 0.65 |
| BW change 1-90 DIM | −104.42 | −119.08 | 11.00 | 0.21 | — | — |

^{a}Values with different superscripts differ, P < 0.05.
^{×}Values with different superscripts tend to differ, P <0.10
¹LG = leaky gut

Milk yield was improved on average by 5.8 lbs per cow per day in the first 90 days of lactation with supplementing the leaky gut product in the feed. (Table 2). There was no difference among treatments in body condition score (BCS) or body weight (BW) change throughout the trial. The cows consuming the leaky gut product also did not eat more pellet, which may indicate an improvement in feed efficiency.

TABLE 24
Milk component and energy-corrected milk yields.

P -value

| | | LG | | | | |
|---|---|---|---|---|---|---|
| Item | Control | Product | SE | Trt | Week | Trt*Wk |
| Fat Yield, lbs | 4.65 | 4.97 | 0.08 | 0.0052 | <0.0001 | 0.51 |
| Protein Yield, lbs | 3.48 | 3.60 | 0.06 | 0.17 | <0.0001 | 0.15 |
| Component Yield, lbs | 8.11 | 8.56 | 0.12 | 0.0099 | <0.0001 | 0.52 |
| ECM Yield, lbs | 122.22 | 130.21 | 1.87 | 0.0025 | <0.0001 | 0.73 |

It is desirable to not only increase milk yield but also increase the yield of fat and protein. Energy-corrected milk yield (ECM) is a calculated way to standardize milk for components and is calculated using the following equation: (0.327*Milk Yield)+(12.95*Fat Yield)+(7.2*Protein Yield). Supplementing LG product in the feed of lactating dairy cows before expected calving date through 90 days in lactation resulted in 0.32 lbs of more milk fat produced per cow per day, 0.12 lbs more milk protein produced per cow per day, and 8 lbs of ECM yield produced per cow per day (Table 3).

### Example 8

TABLE 25
Impact of LG product on finisher performance.

| | | LG | | Control vs LG |
|---|---|---|---|---|
| | Control | Product¹ | SEM | Product |
| Number of Pigs | 306 | 276 | | |
| Start BW², lb | 128.7 | 129.0 | 3.17 | 0.672 |

Growth Performance
d 0-13

| | | | | |
|---|---|---|---|---|
| ADG³, lb/day | 1.50 | 1.55 | 0.019 | 0.079 |
| ADFI⁴, lb/day | 2.13 | 2.66 | 0.034 | 0.001 |
| Final BW, lb | 69.1 | 70.0 | 0.27 | 0.020 |

d 14-27

| | | | | |
|---|---|---|---|---|
| ADG, lb/day | 2.21^{b} | 2.32^{a} | 0.028 | 0.006 |
| ADFI, lb/day | 6.23^{b} | 6.40^{a} | 0.051 | 0.023 |
| BW on d 27, lb | 190.1^{b} | 192.4^{a} | 0.55 | 0.006 |

d 27-41

| | | | | |
|---|---|---|---|---|
| ADG, lb/day | 2.23 | 2.22 | 0.039 | 0.857 |
| ADFI, lb/day | 6.11^{b} | 6.58^{a} | 0.108 | 0.004 |
| BW on d 41, lb | 221.6 | 223.7 | 0.93 | 0.103 |

d 41-55

| | | | | |
|---|---|---|---|---|
| ADG, lb/day | 1.74 | 1.82 | 0.045 | 0.215 |
| ADFI, lb/day | 6.24 | 6.20 | 0.101 | 0.721 |
| BW on d 55, lb | 245.0 | 248.4 | 1.19 | 0.049 |

d 55-74

| | | | | |
|---|---|---|---|---|
| ADG, lb/day | 2.15 | 2.08 | 0.038 | 0.228 |
| ADFI, lb/day | 6.99 | 7.17 | 0.094 | 0.156 |
| BW on d 74, lb | 282.0 | 285.0 | 1.51 | 0.167 |

d 0-74

| | | | | |
|---|---|---|---|---|
| ADG, lb/day | 2.13 | 2.16 | 0.017 | 0.300 |
| ADFI, lb/day | 6.26^{b} | 6.43^{a} | 0.055 | 0.023 |
| Closeout ADG, lb/day | 2.07 | 2.06 | 0.035 | 0.820 |

¹LG product includes green coffee bean at 0.05 lb/ton and a blend of direct-fed microbials provided at 500,000 CFU's per gram of feed.
²BW = body weight
³ADG = average daily gain
⁴ADFI = average daily feed intake

A total of 582 finishing gilts and barrows were used to evaluate the dietary inclusion of the LG product for finishing pigs. All animals were fed diets formulated with nutrients that met or exceeded the animals requirement for economic growth and feed conversion. Diets for the animals supplemented with the LG product were included at the expense of ground corn in the formula. Supplementing LG product in the feed of finishing pigs with a starting body weight of 129 lbs resulted in improved average daily gain, body weight and improved feed intake during the 74-day period the product was fed. The differences observed varied by time period of measurement. Supplementation of LG product, improved ADG by 0.05 lbs, feed intake by 0.53 lbs, and final BW by 0.9 lbs. From day 14-27, ADG was improved by 0.11 lbs, feed intake increased by 0.17 lbs, and BW at 27 days increased by 2.30 lbs. Additionally, from day 27-41, feed intake increased by 0.47 lbs, and from day 41-55 BW improved by 3.40 lbs. with supplementation of the LG product. Feed intake increased by 0.18 lbs from day 55-74 and by 0.17 lbs over the entire duration of the trial.

What is claimed is:

1. A feed composition comprising:
a therapeutically effective quantity of at least one strain of bacteria in the Bacillus genus;
a therapeutically effective quantity of chlorogenic acid.

2. The composition of claim 1, wherein the species of Bacillus is selected from the group consisting of: Bacillus amyloliquefaciens, Bacillus subtilis, Bacillus velezensis or a combination thereof.

3. The composition of claim 2, wherein the strain of Bacillus is selected from the group consisting of: BA22, BA1013, ZB617, CD1718, CGS1.1, or a combination thereof.

4. The composition of claim 1, wherein the chlorogenic acid is derived from green coffee beans or a coffee-based product.

5. The composition of claim 4, wherein the chlorogenic acid is in the form of green coffee bean extract.

6. The composition of claim 1, wherein:
a. the species of Bacillus is selected from the group consisting of: Bacillus amyloliquefaciens, Bacillus subtilis, Bacillus velezensis or a combination thereof;
b. The concentration of Bacillus is 1×10⁴-3×10¹⁰ CFU/head/day;
c. The chlorogenic acid is in the form of green coffee bean extract; and
d. the range for the green coffee bean extract is 0.005-5 g/head/day.

7. The composition of claim 6, wherein the strain of Bacillus is selected from the group consisting of: BA22, BA1013, ZB617, CD1718, CGS1.1, or a combination thereof.

8. The composition of claim 6, wherein the concentration range for Bacillus is 5.0×10⁵-2×10⁹ CFU/head/day and for green coffee bean extract is 6-500 mg/head/day.

9. A method of improving the health of a non-human animal, the method comprising:
administering to the non-human animal a therapeutically effective amount of a composition comprising:
a therapeutically effective quantity of Bacillus; and
a therapeutically effective quantity of chlorogenic acid;
wherein the administration of the composition improves overall health in a non-human animal.

10. The method of claim 9, wherein the species of Bacillus is selected from the group consisting of: Bacillus amyloliquefaciens, Bacillus subtilis, Bacillus velezensis or a combination thereof.

11. The method of claim 10, wherein the strain of Bacillus is selected from the group consisting of: BA22, BA1013, ZB617, CD1718, CGS1.1, or a combination thereof.

12. The method of claim 9, wherein the overall health improvement in a non-human animal is selected from a list comprising: increasing weight gain, increasing feed conversion, improving intestinal integrity, reducing inflammation, improving milk yield, improving milk quality, reducing mortality rate, reducing the need for using conventional antibiotics, improving feed efficiency, or a combination herein.

13. A method of improving the health of a non-human animal, the method comprising:
administering to a non-human animal a therapeutically effective amount of a composition comprising:
a therapeutically effective quantity of Bacillus; and
a therapeutically effective quantity of chlorogenic acid;
wherein the administration of the composition follows a stressor event.

14. The method of claim 13, wherein the administration of the composition alleviates at least one negative effect of the stressor event.

15. The method of claim 14, wherein the species of Bacillus is selected from the group consisting of: Bacillus amyloliquefaciens, Bacillus subtilis, Bacillus velezensis or a combination thereof.

16. The method of claim 15, wherein the strain of Bacillus is selected from the group consisting of: BA22, BA1013, ZB617, CD1718, CGS1.1, or a combination thereof.

17. The method of claim 14, wherein the alleviation of at least one negative effect of the stressor event is selected from a list comprising: increasing weight gain, increasing feed conversion, improving intestinal integrity, reducing inflammation, improving milk yield, reducing mortality rate, reducing the need for using conventional antibiotics, improving feed efficiency, or a combination herein.

18. The method of any one of claim 14, wherein the stressor event is selected from a list comprising: bacterial infection, viral infection, heat stress, abrupt diet changes, transportation stress, weaning, overcrowding, poor handling, feed restrictions, environmental changes, nutritional deficiencies, chronic stress, dysbiosis, off-feed events, livestock management malpractice, or a combination herein.
